(19) United States
(12) Patent Application Publication
(10) Pub. No.: US 2025/0049679 A1
(43) Pub. Date: Feb. 13, 2025

(54) FORMULATIONS OF ERIODICTYOL

(71) Applicant: DEBUT BIOTECHNOLOGY, INC., San Diego, CA (US)

(72) Inventor: Taylor Oswald, San Diego, CA (US)

(21) Appl. No.: 18/799,164

(22) Filed: Aug. 9, 2024

Related U.S. Application Data
(60) Provisional application No. 63/531,603, filed on Aug. 9, 2023.
(60) Provisional application No. 63/531,606, filed on Aug. 9, 2023.

(51) Int. Cl. A61K 8/49; A61K 8/06; A61K 9/00; A61K 9/107; A61K 31/353; A61Q 19/08

(52) U.S. Cl. CPC A61K 8/498; A61K 8/062; A61K 9/0014; A61K 9/107; A61K 31/353; A61Q 19/08

(57) ABSTRACT

The invention provides compositions comprising eriodictyol. The compositions of the invention may be administered topically, on the skin of the subject. The invention further provides methods of administration of compositions comprising eriodictyol for amelioration or treatment of a skin condition. The compositions of the invention may also be applied for improving the general appearance of the skin.

### I. FIELD OF THE INVENTION

The invention is related to formulations of eriodictyol and methods of using the formulations for topical conditions in a subject.

### II. BACKGROUND OF THE INVENTION

Mammalian epidermis and its appendages (e.g., hair, nail, sebaceous and sweat glands) provide a barrier to keep harmful elements out of the body and essential body fluids in. As the first line of defense against the various physical traumas of the environment, the epidermis must protect itself as well as the underlying tissues. The epidermis is also exposed to mutagenic ultraviolet radiation. In non-haired or sparsely haired regions such as most human skin, the epidermis is thicker than that of furred skin, and in these locations the skin functions primarily in a protective role. The constant assaults on the epidermis necessitate self-renewal, making the epidermis a prime example of an adult tissue that undergoes continual and rapid flux.

Aged skin differs from youthful skin both in appearance and in function. The aged epidermis demonstrates decreased keratinocyte proliferation and is physically thinner, but mostly from decline of the rete ridges. In addition to thinning, aging slows wound healing, prolongs epidermal turnover, and impairs barrier formation. The skin appears thin, wrinkled, bruised, and rough. Wrinkling and bruising can also result from aging-related changes in the dermis. The dermis, too, is characteristically thinner in aged persons. Aged fibroblasts are less likely to synthesize normal amounts of collagen, elastin, laminin glycosaminoglycans, and fibronectin. The dermis can, in such cases, lack elasticity, strength, vessel support, remodeling abilities, and ground substances. For example, rete ridges can be effaced, and basal cells can no longer display villous projections into the dermis. Epidermal cell turnover is reduced up to 50% in the aged as compared to youth.

For example, melanocyte numbers can decrease 8-20% per decade. There are fewer Langerhans cells in the aged, and those present are often functionally impaired. Collagen synthesis decreases, for example, up to 30% within 4 years of menopause in women. The numbers of collagen and elastic fibers are also decreased. The dermis can also become less echogenic to ultrasounds, consistent with changes in collagen and elastic tissues.

### III. SUMMARY OF THE INVENTION

The invention relates to compositions of eriodictyol, and methods of using the compositions for treating, protecting, and/or altering (e.g., improving) the condition and/or aesthetic appearance of skin, including, for example, treating, preventing, ameliorating, reducing and/or eliminating fine lines and/or wrinkles of skin and/or improving the aesthetic appearance of fine lines and/or wrinkles of skin. In certain embodiments, the compositions of the invention lead to increase in skin and/or hair resiliency. In certain embodiments, the compositions of the invention lead to increase in elasticity of the skin. In certain embodiments, the compositions of the invention prevent skin damage. In certain embodiments, the compositions of the invention prevent the formation of scar tissue in the process of wound healing.

Topical administration of compositions comprising eriodictyol were not known in the field for application on human skin. In particular, the topical administration of eriodictyol for improving the condition of skin, including enhancing the appearance of the skin, by topical administration of a composition comprising an effective amount of eriodictyol was not known in the field. Beneficially, the invention recognizes, for the first time, that topical compositions comprising eriodictyol may be used for improving the condition of the skin or for treatment of any ailment related to the skin of a subject.

In one aspect, the invention provides a composition for topical administration comprising an effective amount of eriodictyol. The effective amount of eriodictyol is an amount of eriodictyol that would be effective in treating, ameliorating, or otherwise impacting the underlying condition of the skin. The invention recognizes that eriodictyol is poorly soluble or insoluble in water. The invention also recognizes that eriodictyol is also poorly soluble or insoluble in oil, for example, eriodictyol is insoluble in Helianthus Annuus (Sunflower) Seed Oil. In addition, eriodictyol is also only minimally soluble in pentylene glycol and capric/caprylic triglyceride. In certain embodiments of the invention, eriodictyol is dissolved in a solvent selected from a group consisting of: dimethyl isosorbide, ethoxydiglycol, isopropyl lauroyl sarcosinate, and any combinations thereof. In certain preferred embodiments, eriodictyol is dissolved in dimethyl isosorbide. In certain embodiments, the composition comprising eriodictyol is anhydrous. In certain embodiments, the composition comprises oil as the only solvent.

In certain embodiments, the composition comprising eriodictyol is an emulsion. In certain embodiments, the emulsion is an oil/water emulsion. In certain embodiments, the composition further comprises a solvent. In certain embodiment, the solvent is selected from a group consisting of: dimethyl isosorbide, ethoxydiglycol, isopropyl lauroyl sarcosinate, and any combinations thereof. In certain embodiments, the solvent in the composition is dimethyl isosorbide. In certain embodiments, the solvent in the composition is ethoxydiglycol. In certain embodiments, the solvent in the composition is isopropyl lauroyl sarcosinate. In certain embodiments, the solvent in the composition is a glycol. In certain embodiments, the glycol is selected from a group consisting of pentylene glycol, butylene glycol, propylene glycol, and any combination thereof.

In certain embodiments, eriodictyol is dissolved in a solvent selected from a group consisting of: 1,2 heptanediol, 1,2 hexanediol, 1,2 propanediol, butylene glycol, C₁₅₋₁₉ alkane, capric/caprylic triglyceride, dibutyl adipate, diisopropyl adipate, dimethyl isosorbide, dipropylene glycol, ethanol, ethoxydiglycol, isoamyl laurate, isohexadecane, isopentyldiol, isopropyl myristate, isoproyl lauroyl sarcosinate, jojoba oil, meadowfoam seed oil, mineral oil, neopentyl glycol diethylhexanoate, octyldodecanol, oleyl alcohol, pentylene glycol, phenylpropanol, polysorbate 20, propanediol, safflower seed oil, squalane, squalene, sunflower oil, t-butyl alcohol, tributyl citrate, tributyl-O-acetylcitrate, tri-C₁₅₋₁₉ alkyl citrate, triethyl citrate, triethyl O-acetylcitrate, and triisostearin.

The compositions of the invention are stable under storage in ambient conditions. This is beneficial because the compositions of the invention may be stored prior to administration to the subjects. There were no previously known shelf-stable formulations of eriodictyol. Shelf-storage stable formulations of eriodictyol would provide a convenient option for the composition to be applied to patients suffering from various ailments of the skin and/or seeking options for appearance of the skin.

In certain preferred embodiments, the composition of the invention, comprising eriodictyol, are stable under storage for at least two (2) years under ambient conditions. In certain embodiments, the composition of the invention, comprising eriodictyol, are stable under storage for at least one year under ambient conditions. In certain embodiments, the composition of the invention, comprising eriodictyol, are stable under storage for at least six (6) months under ambient conditions. In certain embodiments, the composition of the invention, comprising eriodictyol, are stable under storage for at least three (3) months under ambient conditions.

In certain embodiments, the composition demonstrates less than about 5% degradation when stored at 50° C. for one (1) month. In certain embodiments, the composition demonstrates less than about 10% degradation when stored at 50° C. for one (1) month. In certain embodiments, the composition demonstrates less than about 10% degradation when stored at 40° C. for one (1) month. In certain embodiments, the composition demonstrates less than about 1% degradation when stored at 25° C. and/or 4° C. for one (1) month. In certain embodiments, the composition demonstrates less than about 10% degradation when stored at 40° C. for three (3) months. In certain embodiments, the composition demonstrates less than about 10% degradation when stored at 25° C. for three (3) months. In certain embodiments, the composition demonstrates less than about 10% degradation when stored at 25° C. for six (6) months. In certain embodiments, the composition demonstrates less than about 5% degradation when stored at 4° C. for three (3) months.

The high stability of the compositions of the invention under the conditions of accelerated stability demonstrate that the compositions of the invention will be stable under ambient conditions for at least 2 years. In certain embodiments, the compositions comprise eriodictyol dissolved in dimethyl isosorbide, isopropyl lauroyl sarcosinate, and ethoxydiglycol.

Surprisingly, the stability of eriodictyol is dependent on the pH of the composition. The invention recognizes that eriodictyol degrades at a higher rate if the pH of the composition is not in an optimal pH range. Thus, in certain embodiments, the compositions of the invention comprise buffers and/or pH adjusting agents to maintain the pH of the composition in an optimal range to minimize the degradation of eriodictyol under ambient conditions. In certain embodiments, the pH of the composition is from about 4 to about 8. In certain embodiments, the pH of the composition is from about 4 to about 7. In certain embodiments, the pH of the composition is from about 4 to about 6. In certain embodiments, the pH of the composition is from about 4 to about 5. The data pertaining to stability of the compositions of the invention with respect to the pH of the solution is provided in FIG. 1.

In certain embodiments, the pH of the composition is about 4. In certain embodiments, the compositions comprising eriodictyol and a pH of about 4 have less than about 10% degradation when stored at 50° C. for one (1) month.

In certain embodiments, the pH of the composition is about 5. In certain embodiments, the compositions comprising eriodictyol and a pH of about 5 have less than about 10% degradation when stored at 50° C. for one (1) month. In certain embodiments, the compositions comprising eriodictyol and a pH of about 5 have less than about 20% degradation when stored at 50° C. for one (1) month.

In certain embodiments, the pH of the composition is about 6. In certain embodiments, the compositions comprising eriodictyol and a pH of about 6 have less than about 10% degradation when stored at 50° C. for one (1) month. In certain embodiments, the compositions comprising eriodictyol and a pH of about 6 have less than about 20% degradation when stored at 50° C. for one (1) month.

In certain embodiments, the pH of the composition is about 7. In certain embodiments, the compositions comprising eriodictyol and a pH of about 7 have less than about 20% degradation when stored at 50° C. for one (1) month. In certain embodiments, the compositions comprising eriodictyol and a pH of about 6 have less than about 25% degradation when stored at 50° C. for one (1) month.

In certain embodiments, the compositions of the inventions comprise eriodictyol in a concentration of about 5 nM to about 5 mM. In certain embodiments, the compositions of the inventions comprise eriodictyol in a concentration of about 10 nM to about 4 mM. In certain embodiments, the compositions of the invention comprise eriodictyol in a concentration of about 5 nM to about 2500 nM. In certain embodiments, the compositions of the invention comprise eriodictyol in a concentration of about 10 nM to about 2000 nM. In certain embodiments, the compositions of the invention comprise eriodictyol in a concentration of about 20 nM to about 1000 nM. In certain embodiments, the compositions of the invention comprise eriodictyol in a concentration of about 10 nM to about 20 nM. In certain embodiments, the compositions of the inventions comprise eriodictyol in a concentration of about 5 μM to about 3,750 μM. In certain embodiments, the compositions of the inventions comprise eriodictyol in a concentration of about 50 μM to about 3,000 μM. In certain embodiments, the compositions of the inventions comprise eriodictyol in a concentration of about 50 μM to about 3,000 μM.

In certain embodiments, eriodictyol is present in a concentration of about 0.0001% to about 5.0% w/w in the compositions of the invention. In certain embodiments, eriodictyol is present in a concentration of about 0.0001% to about 1.0% w/w in the compositions of the invention. In certain embodiments, eriodictyol is present in a concentration of about 0.001% to about 0.5% w/w in the compositions of the invention. In certain embodiments, eriodictyol is present in a concentration of about 0.01% to about 0.5% w/w in the composition of the invention. In certain embodiments, eriodictyol is present in a concentration of about 0.1% to about 0.5% w/w in the compositions of the invention. In certain embodiments, eriodictyol is present in a concentration of about 0.5% w/w in the compositions of the invention.

In certain aspects, the compositions of the invention may be administered as a topical composition on the skin of the subject. The compositions of the invention may be included in any formulation suitable for topical administration. In certain embodiments, the composition of the invention is a fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, and/or hair treatment. In certain embodiments, the compositions of the invention also include excipients. These excipients may be selected from a group consisting of: solvent, emulsifier, preservative, antioxidant, emollient, thickening agent, penetration enhancer, surfactant, diluent, filler, carrier, and/or pH control agent. In certain embodiments, the preservative in the composition is an antimicrobial preservative or chelating agent. In certain embodiments, the excipients in the composition are selected from the group consisting of: dimethyl isosorbide, ethoxydiglycol, glycol, isopropyl lauroyl sarcosinate, 1,2 hexanediol, propanediol, phenylpropanol, isopentyldiol, 1,2 heptanediol, water, glycerin, hydrogenated ethylhexyl olivate, hydrogenated olive oil unsaponifiables, polyglycerol-6-distearate, jojoba esters, polyglyceryl-3-beeswax, cetyl alcohol, cetearyl olivate, sorbitan olivate, hydroxyethyl acrylate/sodium acryloyldimethyl taurate copolymer, Helianthus annuus (sunflower) seed oil, caprylic/capric triglyceride, phenoxyethanol, decylene glycol, and 1,2-hexanediol.

The compositions of the invention may be prepared by various methods. As an example, the compositions may be prepared by dissolving, dispersing, etc. eriodictyol as provided herein and optional excipients in a carrier (e.g., water, saline, aqueous dextrose, glycerol, glycols, ethanol or the like) or a solvent to form a solution, colloid, liposome, emulsion, complexes (including inclusion complexes), coacervate, or suspension. In certain embodiments, the compositions of the current invention can also contain additional excipients such as wetting agents, emulsifying agents, co-solvents, solubilizing agents, pH buffering agents, and the like (e.g., sodium acetate, sodium citrate, cyclodextrins and derivatives, sorbitan monolaurate, triethanolamine acetate, triethanolamine oleate, hydroxyethylpiperazine and the like). In certain embodiments, the compositions of the invention may further include one or more additional excipients selected from a group consisting of: carriers, excipients, or diluents including, but not limited to, absorbents, anti-irritants, antibacterials, anti-acne agents, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

In certain embodiments, the compositions of the invention comprise: eriodictyol, in an oil/water emulsion, and a preservative.

In certain embodiments, the compositions of the invention comprise: eriodictyol, in an oil/water emulsion, a solvent for solubilizing eriodictyol, and a preservative.

In certain embodiments, the compositions of the invention comprise eriodictyol, in an oil/water emulsion, a solvent for solubilizing eriodictyol, a diluent, and a preservative.

In certain embodiments, the compositions of the invention comprise: eriodictyol, in an oil/water emulsion, a solvent for solubilizing eriodictyol, a thickener, optionally a diluent, and a preservative.

In certain embodiments, the compositions of the invention comprise: eriodictyol, in an oil/water emulsion, a solvent for solubilizing eriodictyol, and one or more excipients selected from the group consisting of: diluents, absorbents, antioxidants, emollients, emulsifiers, thickeners, surfactants, and/or preservatives.

In certain embodiments, the excipients included in the composition are water, dimethyl isosorbide, glycerin, caprylic/capric triglyceride, propanediol, sunflower seed oil, ethoxydiglycol, squalane, sodium acrylate copolymer, cetearyl alcohol, phenoxyethanol, glyceryl stearate, capryloyl glycerin/sebacic acid copolymer, diheptyl succinate, naringenin, lecithin, decylene glycol, pentylene glycol, cetearyl glucoside, shea butter, palmitic acid, arachidyl alcohol, behenyl alcohol, arachidyl glucoside, phenethyl alcohol, 1,2-hexanediol, hydrogenated olive oil, olea europaea (olive) fruit oil, Olea europaea (olive) oil unsaponifiables, hydroxyethyl acrylate/sodium acryloyldimethyl taurate copolymer, citric acid, sodium acetylated hyaluronate, sodium hyaluronate crosspolymer, sodium phytate, hydrolyzed sodium hyaluronate, and/or ethylhexylglycerin.

In certain aspects of the invention, the compositions comprising eriodictyol demonstrate antioxidant activity. In certain embodiments, the compositions of the invention, the antioxidant activity of eriodictyol is demonstrated at concentrations of about 0.00016% w/w or higher. Beneficially, the antioxidant activity of compositions comprising eriodictyol is demonstrated in both aqueous and organic solvent systems. Thus, the invention provides compositions which retain antioxidant activity in both aqueous and organic solvents. Advantageously, the compositions of the invention comprising eriodictyol, demonstrating antioxidant activity, are effective for treating and/or preventing oxidation damage. In certain embodiments, the compositions of the invention comprising eriodictyol, demonstrating antioxidant activity, are effective for treatment of skin and/or hair damaged by reactive oxygen species and protection from other environmental aggressors that may damage the skin of the subject. In certain other embodiments, the compositions of the invention comprising eriodictyol, demonstrating antioxidant activity, are effective for prevention of skin and/or hair damage by reactive oxygen species, including the damage caused by environmental aggressors.

In certain aspects of the invention, the compositions comprising eriodictyol demonstrate elastase enzyme inhibition. In certain embodiments, the compositions of the invention, the elastase enzyme inhibition activity of eriodictyol is demonstrated at concentrations of about 0.008% w/w or higher. Advantageously, the compositions comprising eriodictyol, demonstrating elastase enzyme inhibition activity, are effective for promoting the elasticity of the skin. In certain embodiments, the compositions of the invention comprising eriodictyol, demonstrating elastase enzyme inhibition, are effective in reduction of fine lines and/or wrinkles in the skin. In certain embodiments, the compositions of the invention comprising eriodictyol, demonstrating elastase enzyme inhibition, are effective in prevention of formation of fine lines and/or wrinkles in the skin.

In certain aspects of the invention, the compositions comprising eriodictyol demonstrate inhibition of glycation. In certain embodiments, the compositions of the invention, the glycation inhibition activity of eriodictyol is demonstrated at concentrations of about 0.002% w/w or higher. Advantageously, the compositions comprising eriodictyol, demonstrating glycation inhibition activity, are effective for supporting and promoting skin and/or hair resiliency.

In certain embodiments of the invention, the compositions of the invention, comprising eriodictyol modulate the gene expression of one or more genes that is related to a topical condition. In certain embodiments, administration of composition comprising eriodictyol alters one or more of a function related with the modulated genes, wherein the one or more function is selected from the group consisting of: (i) formation of fines lines or wrinkles on the skin, (ii) inflammation, (iii) skin elasticity, (iv) prevention of scar formation upon wound healing, and/or (v) any combination thereof.

In certain aspects, the invention provides methods of treatment of a topical condition by administration of the topical composition comprising an effective amount of eriodictyol. In certain embodiments, the invention provides methods for prevention of fine lines or wrinkles on skin of the subject by administration of the compositions provided herein. In certain embodiments, the invention provides methods for supporting firmness and increasing/maintaining elasticity of skin by administration of the compositions provided herein. In certain embodiments, the invention provides methods for prevention of skin and hair damaged by reactive oxygen species by administration of the compositions provided herein. In certain embodiments, the invention provides methods for protection from environment aggressors on skin by administration of the compositions provided herein. In certain embodiments, the invention provides methods for increase in skin and/or hair resiliency by administration of the compositions provided herein. In certain embodiments, the invention provides methods for prevention of glycation by administration of the compositions provided herein. Glycation is known to be linked with the wrinkling, loss of elasticity, and aging in the skin. In certain embodiments, the invention provides methods of reducing damage in skin by administration of compositions provided herein.

In certain embodiments, administration of composition comprising eriodictyol alters expression and/or function of one or more genes are selected from a group consisting of: MMP10, THBS1, TOP2A, TFPI2, MMP1, FN1, KCTD4, ATP12A, ALDH1A3, MKI67, FST, SLC13A5, IL6, IL23A, TNF, IL24, MMP3, MMP9, and/or PPFIA4.

In certain embodiments, administration of composition comprising eriodictyol leads to down-regulation of expression and/or activity of MMP10, THBS1, TOP2A, TFPI2, MMP1, FN1, KCTD4, ATP12A, ALDH1A3, MKI67, FST, SLC13A5, IL6, IL23A, TNF, IL24, MMP3, and/or MMP9.

In certain embodiments, the administration of the composition leads to up-regulation of expression and/or activity of PPFIA4.

In certain embodiments, the administration of the composition enhances anti-inflammatory activity. In certain embodiments, the anti-inflammatory activity of the composition is modulated through the downregulation of expression and/or activity of IL6, IL23a, TNF, and/or IL24.

In certain embodiments, the administration of the composition leads to anti-aging activity. In certain embodiments, the anti-aging activity of the composition comprising eriodictyol is modulated through downregulation of expression and/or activity of MMP10, MMP9, MMP3, and MMP1.

In certain embodiments, the administration of the composition comprising eriodictyol enhances maintenance of skin barrier lipids. In certain embodiments, the maintenance of skin barrier lipids is modulated by upregulation of expression and/or activity of PPFIA4.

In certain embodiments, the administration of the composition comprising eriodictyol results in prevention of formation of scar tissue after healing of wounds. In certain embodiments, the prevention of formation of scar tissue after healing of wounds is modulated by downregulation of expression and/or activity of THBS1.

In certain embodiments, the administration of the composition comprising eriodictyol leads to at least about ten (10) fold downregulation of the activity and/or expression of matrix metallopeptidase 10 (MMP10). In certain embodiments, administration of the composition comprising eriodictyol leads to about twenty-five (25) fold downregulation of the activity and/or expression of matrix metallopeptidase 10 (MMP10).

In certain embodiments, administration of the composition comprising eriodictyol leads to at least about ten (10) fold downregulation of the activity and/or expression of thrombospondin 1 (THBS1). In certain embodiments, administration of the composition comprising eriodictyol leads to about fifteen (15) fold downregulation of the activity and/or expression of thrombospondin 1 (THBS1).

In certain embodiments, administration of the composition comprising eriodictyol leads to at least about ten (10) fold downregulation of the activity and/or expression of DNA topoisomerase II-α (TOP2A). In certain embodiments, administration of the composition comprising eriodictyol leads to about fifteen (15) fold downregulation of the activity and/or expression of DNA topoisomerase II-α (TOP2A). In certain embodiments, administration of the composition comprising eriodictyol leads to about twenty (20) fold downregulation of the activity and/or expression of DNA topoisomerase II-α (TOP2A).

In certain embodiments, wherein the administration of the composition comprising eriodictyol leads to at least about ten (10) fold downregulation of the activity and/or expression of tissue factor pathway inhibitor 2 (TFPI2). In certain embodiments, administration of the composition comprising eriodictyol leads to about fifteen (15) fold downregulation of the activity and/or expression of tissue factor pathway inhibitor 2 (TFPI2). In certain embodiments, administration of the composition comprising eriodictyol leads to about twenty (20) fold downregulation of the activity and/or expression of tissue factor pathway inhibitor 2 (TFPI2).

In certain embodiments, administration of the composition comprising eriodictyol leads to at least about two (2) fold downregulation of the activity and/or expression of matrix metallopeptidase 1 (MMP1). In certain embodiments, administration of the composition comprising eriodictyol leads to about ten (10) fold downregulation of the activity and/or expression of matrix metallopeptidase 1 (MMP1).

In certain embodiments, administration of the composition leads to at least about two (2) fold downregulation of the activity and/or expression of fibronectin 1 (FN1). In certain embodiments, administration of the composition leads to about ten (10) fold downregulation of the activity and/or expression of fibronectin 1 (FN1).

In certain embodiments, administration of the composition leads to at least about five (5) fold downregulation of the activity and/or expression of potassium channel tetramerization domain containing 4 (KCTD4). In certain embodiments, administration of the composition leads to about fifteen (15) fold downregulation of the activity and/or expression of potassium channel tetramerization domain containing 4 (KCTD4).

In certain embodiments, administration of the composition comprising eriodictyol leads to at least about two (2) fold downregulation of the activity and/or expression of ATPase H+/K+ transporting non-gastric α2 subunit (ATP12A). In certain embodiments, administration of the composition comprising eriodictyol leads to about ten (10) fold downregulation of the activity and/or expression of ATPase H+/K+ transporting non-gastric α2 subunit (ATP12A).

In certain embodiments, administration of the composition comprising eriodictyol leads to at least about two (2) fold downregulation of the activity and/or expression of aldehyde dehydrogenase 1 family member A3 (ALDH1A3). In certain embodiments, administration of the composition comprising eriodictyol leads to about ten (10) fold downregulation of the activity and/or expression of aldehyde dehydrogenase 1 family member A3 (ALDH1A3).

In certain embodiments, administration of the composition comprising eriodictyol leads to at least about two (2) fold downregulation of the activity and/or expression of marker of proliferation Ki-67 (MKI67). In certain embodiments, administration of the composition comprising eriodictyol leads to about ten (10) fold downregulation of the activity and/or expression of marker of proliferation Ki-67 (MK167).

In certain embodiments, administration of the composition comprising eriodictyol leads to at least about two (2) fold downregulation of the activity and/or expression of follistatin (FST). In certain embodiments, administration of the composition comprising eriodictyol leads to about ten (10) fold downregulation of the activity and/or expression of follistatin (FST).

In certain embodiments, administration of the composition comprising eriodictyol leads to at least about five (5) fold downregulation of the activity and/or expression of solute carrier family 13 member 5 (SLC13A5). In certain embodiments, administration of the composition comprising eriodictyol leads to about fifteen (15) fold downregulation of the activity and/or expression of solute carrier family 13 member 5 (SLC13A5).

In certain embodiments, administration of the composition comprising eriodictyol leads to at least about five (5) fold downregulation of the activity and/or expression of interleukin 6 (IL6). In certain embodiments, administration of the composition comprising eriodictyol leads to about twenty (20) fold downregulation of the activity and/or expression of interleukin 6 (IL6).

In certain embodiments, administration of the composition comprising eriodictyol leads to at least about two (2) fold downregulation of the activity and/or expression of interleukin 23 (IL23). In certain embodiments, administration of the composition comprising eriodictyol leads to about five (5) fold downregulation of the activity and/or expression of interleukin 23 (IL23).

In certain embodiments, administration of the composition comprising eriodictyol leads to at least about ten (10) fold downregulation of the activity and/or expression of tumor necrosis factor (TNF). In certain embodiments, administration of the composition comprising eriodictyol leads to about two hundred fifty (250) fold downregulation of the activity and/or expression of tumor necrosis factor (TNF).

In certain embodiments, administration of the composition comprising eriodictyol leads to at least about ten (10) fold downregulation of the activity and/or expression of interleukin 24 (IL24). In certain embodiments, administration of the composition comprising eriodictyol leads to about seventy (70) fold downregulation of the activity and/or expression of interleukin 24 (IL24).

In certain embodiments, administration of the composition comprising eriodictyol leads to at least about ten (10) fold downregulation of the activity and/or expression of matrix metallopeptidase 3 (MMP3). In certain embodiments, administration of the composition comprising eriodictyol leads to about fifty (50) fold downregulation of the activity and/or expression of matrix metallopeptidase 3 (MMP3).

In certain embodiments, administration of the composition comprising eriodictyol leads to at least about two (2) fold downregulation of the activity and/or expression of matrix metallopeptidase 9 (MMP9). In certain embodiments, administration of the composition comprising eriodictyol leads to about eight (8) fold downregulation of the activity and/or expression of matrix metallopeptidase 9 (MMP9).

In certain embodiments, administration of the composition comprising eriodictyol leads to at least about five (5) fold upregulation of the activity and/or expression of stimulated by PTPRF interacting protein α4 (PPFIA4). In certain embodiments, administration of the composition comprising eriodictyol leads to about thirty (30) fold upregulation of the activity and/or expression of PTPRF interacting protein α4 (PPFIA4).

### IV. BRIEF DESCRIPTION OF THE DRAWINGS

FIG. 1 provides the stability data of compositions comprising eriodictyol.

### V. DETAILED DESCRIPTION

### Eriodictyol

Eriodictyol is a naturally occurring polyphenol primarily found in the yerba Santa Clause plant. Venditti et al., Deng et al., Pharmacological Activity of Eriodictyol: The Major Natural Polyphenolic Flavanone, Evidence-Based Complementary and Alternative Medicine, 2020, Article ID 6681352. Eriodictyol, being a well-known natural bitter masker used in the food industry, especially in wine, is expected to have very low or zero toxicity and, to the best of our knowledge, there are no reported toxicity results for eriodictyol.

The invention provides compositions of eriodictyol. The chemical structure of eriodictyol is provided below:

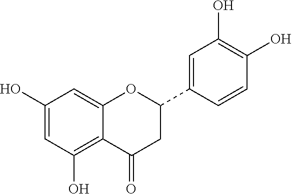 [SMILES: O=C1C[C@@H](c2ccc(O)c(O)c2)Oc2cc(O)cc(O)c21]

Eriodictyol possesses anti-inflammatory activity, which is demonstrated by eriodictyol's ability to decrease inflammatory markers in macrophage cells. Eriodictyol also demonstrates anti-inflammatory activity in mice; alleviating symptoms of atopic dermatitis in mouse skin. Park et al., Effect of eriodictyol on the development of atopic dermatitis-like lesions in ICR mice, Biol Pharm Bull, 2013; 36 (8): 1375-9.

Additionally, eriodictyol reduces neuroinflammation and relieves hypererythema by acting on TRPV1 receptors. Deng et al., Pharmacological Activity of Eriodictyol: The Major Natural Polyphenolic Flavanone, Evidence-Based Complementary and Alternative Medicine, 2020, Article ID 6681352. Neurology studies have been further extended to neurodegenerative disease, where eriodictyol decreases cognitive decline in mice by acting on the Nrf2/HO-1 pathway. Li et al., Eriodictyol ameliorates cognitive dysfunction in APP/PSI mice by inhibiting ferroptosis via vitamin D receptor-mediated Nrf2 activation, Molecular Medicine, 2022, 28, 11. With regards to the skin, eriodictyol decreases melanogenesis in melanoma cells by inhibiting tyrosinase and protecting skin from UVA-induced photodamage and apoptosis. Imen et al., Anti-melanogenesis and antigenotoxic activities of eriodictyol in murine melanoma (B16-F10) and primary human keratinocyte cells, Life Sci., 2015, 135-173; Nakashima et al., Inhibitors of melanogenesis in B16 melanoma 4A5 cells from flower buds of Lawsonia inermis (Henna), Bioorg Med Chem Lett., 2015, 25 (13), 2702-6; Lee et al., The Anti-apoptotic and Anti-oxidant Effect of Eriodictyol on UV-Induced Apoptosis in Keratinocytes, Biol Pharm Bull, 2007 January; 30 (1): 32-7.

Eriodictyol also demonstrates antioxidant, cardioprotective, anti-cancer, anti-diabetic, and hepatoprotective activities of eriodictyol.

### Compositions of Eriodictyol:

In one aspect, the invention provides compositions comprising eriodictyol. In certain embodiments, the invention provides compositions comprising eriodictyol that are suitable for administration on the skin of the subject.

In one aspect, the invention provides a composition for topical administration comprising an effective amount of eriodictyol. The effective amount of eriodictyol is an amount of eriodictyol that would be effective in treating, ameliorating, or otherwise impacting the underlying condition of the skin. The invention recognizes that eriodictyol is poorly soluble or insoluble in water. The invention also recognizes that eriodictyol is also poorly soluble or insoluble in oil, for example, eriodictyol is insoluble in Helianthus Annuus (Sunflower) Seed Oil. In addition, eriodictyol is also only minimally soluble in pentylene glycol and capric/caprylic triglyceride. In certain embodiments of the invention, eriodictyol is dissolved in a solvent selected from a group consisting of: dimethyl isosorbide, ethoxydiglycol, isopropyl lauroyl sarcosinate, and any combinations thereof. In certain preferred embodiments, eriodictyol is dissolved in dimethyl isosorbide. In certain embodiments, the composition comprising eriodictyol is anhydrous. In certain embodiments, the composition comprises oil as the only solvent.

In certain embodiments, eriodictyol is dissolved in a solvent selected from a group consisting of: 1,2 heptanediol, 1,2 hexanediol, 1,2 propanediol, butylene glycol, C₁₅₋₁₉ alkane, capric/caprylic triglyceride, dibutyl adipate, diisopropyl adipate, dimethyl isosorbide, dipropylene glycol, ethanol, ethoxydiglycol, isoamyl laurate, isohexadecane, isopentyldiol, isopropyl myristate, isoproyl lauroyl sarcosinate, jojoba oil, meadowfoam seed oil, mineral oil, neopentyl glycol diethylhexanoate, octyldodecanol, oleyl alcohol, pentylene glycol, phenylpropanol, polysorbate 20, propanediol, safflower seed oil, squalene, sunflower oil, t-butyl alcohol, tributyl citrate, tributyl-O-acetylcitrate, tri-C₁₅₋₁₉ alkyl citrate, triethyl citrate, triethyl O-acetylcitrate, and triisostearin.

Table 1, provided below, includes the solubility data for eriodictyol in various solvents.

TABLE 1
Solubility of eriodictyol

| Solvent | Solubility (% w/w) |
|---|---|
| Ethoxydiglycol | 9.15 |
| Dimethylisosorbide | 8.38 |
| Pentylene Glycol | Not soluble |
| Caprylic/Capric Triglyceride | Not soluble |
| Isopropyl Lauroyl Sarcosinate | 7.73 |
| Helianthus Annuus (Sunflower) Seed Oil | Not soluble |

As evident from the data provided above, eriodictyol is soluble in dimethyl isosorbide, ethoxydiglycol, and isopropyl lauroyl sarcosinate, and any combinations thereof. However, eriodictyol is minimally soluble in a number of other commonly used solvents.

In certain embodiments, the composition comprising eriodictyol is an emulsion. In certain embodiments, the emulsion is an oil/water emulsion. In certain embodiments, the composition further comprises a solvent. In certain embodiment, the solvent is selected from a group consisting of: dimethyl isosorbide, ethoxydiglycol, isopropyl lauroyl sarcosinate, and any combinations thereof. In certain embodiments, the solvent in the composition is dimethyl isosorbide. In certain embodiments, the solvent in the composition is ethoxydiglycol. In certain embodiments, the solvent in the composition is isopropyl lauroyl sarcosinate.

In certain preferred embodiments, the composition of the invention, comprising eriodictyol, are stable under storage for at least two (2) years under ambient conditions. In certain embodiments, the composition of the invention, comprising eriodictyol, are stable under storage for at least one year under ambient conditions. In certain embodiments, the composition of the invention, comprising eriodictyol, are stable under storage for at least six (6) months under ambient conditions. In certain embodiments, the composition of the invention, comprising eriodictyol, are stable under storage for at least three (3) months under ambient conditions.

In certain embodiments, the composition demonstrates less than about 5% degradation when stored at 50° C. for one (1) month. In certain embodiments, the composition demonstrates less than about 10% degradation when stored at 50° C. for one (1) month. In certain embodiments, the composition demonstrates less than about 10% degradation when stored at 40° C. for one (1) month. In certain embodiments, the composition demonstrates less than about 1% degradation when stored at 25° C. and/or 4° C. for one (1) month. In certain embodiments, the composition demonstrates less than about 10% degradation when stored at 40° C. for three (3) months. In certain embodiments, the composition demonstrates less than about 10% degradation when stored at 25° C. for three (3) months. In certain embodiments, the composition demonstrates less than about 10% degradation when stored at 25° C. for six (6) months. In certain embodiments, the composition demonstrates less than about 5% degradation when stored at 4° C. for three (3) months.

The high stability of the compositions of the invention under the conditions of accelerated stability demonstrate that the compositions of the invention will be stable under ambient conditions for at least 2 years. In certain embodiments, the compositions comprise eriodictyol dissolved in dimethyl isosorbide, isopropyl lauroyl sarcosinate, and ethoxydiglycol.

The stability data for eriodictyol in dimethyl isosorbide is provided in Table 2 below:

TABLE 2
Stability Data

| | 30 day % | 90 day % | 180 day % |
|---|---|---|---|
| Temp | remaining | remaining | remaining |
| 4 C. | 104.66 | 97.15 | |
| 25 C. | 100.78 | 91.72 | 90.30 |
| 40 C. | 93.53 | 98.06 | |
| 50 C. | 95.73 | | |

As evident from the data provided in Table 2, the compositions comprising eriodictyol demonstrate good stability and minimal degradation. The representative data provided in Table 2, with eriodictyol dissolved in dimethyl isosorbide, demonstrates the compositions of the invention, comprising eriodictyol are stable when tested under accelerated conditions. Thus, the compositions of the invention will also be stable for storage for at least 2 years under ambient conditions.

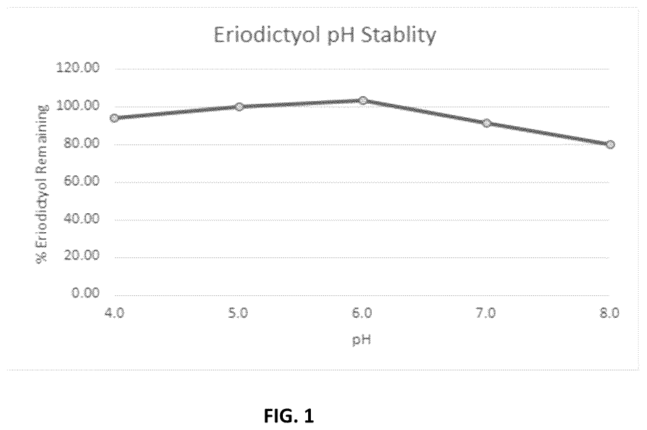

Surprisingly, the stability of eriodictyol is dependent on the pH of the composition. The invention recognizes that eriodictyol degrades at a higher rate if the pH of the composition is not in an optimal pH range. Thus, in certain embodiments, the compositions of the invention comprise buffers and/or pH adjusting agents to maintain the pH of the composition in an optimal range to minimize the degradation of eriodictyol under ambient conditions. In certain embodiments, the pH of the composition is from about 4 to about 8. In certain embodiments, the pH of the composition is from about 4 to about 7. In certain embodiments, the pH of the composition is from about 4 to about 6. In certain embodiments, the pH of the composition is from about 4 to about 5. The data pertaining to stability of the compositions of the invention with respect to the pH of the solution is provided in FIG. 1.

In certain embodiments, the pH of the composition is about 4. In certain embodiments, the compositions comprising eriodictyol and a pH of about 4 have 1 less than about 10% degradation when stored at 50° C. for one (1) month. In certain embodiments, the compositions comprising eriodictyol and a pH of about 4 have 1 less than about 10% degradation when stored at 50° C. for two (2) months.

In certain embodiments, the pH of the composition is about 5. In certain embodiments, the compositions comprising eriodictyol and a pH of about 5 have less than about 10% degradation when stored at 50° C. for one (1) month. In certain embodiments, the compositions comprising eriodictyol and a pH of about 5 have less than about 20% degradation when stored at 50° C. for one (1) month.

In certain embodiments, the pH of the composition is about 6. In certain embodiments, the compositions comprising eriodictyol and a pH of about 6 have less than about 10% degradation when stored at 50° C. for one (1) month. In certain embodiments, the compositions comprising eriodictyol and a pH of about 6 have less than about 20% degradation when stored at 50° C. for one (1) month.

In certain embodiments, the pH of the composition is about 7. In certain embodiments, the compositions comprising eriodictyol and a pH of about 7 have less than about 20% degradation when stored at 50° C. for one (1) month. In certain embodiments, the compositions comprising eriodictyol and a pH of about 6 have less than about 25% degradation when stored at 50° C. for one (1) month.

In certain embodiments, the compositions of the inventions comprise eriodictyol in a concentration of about 5 nM to about 5 mM. In certain embodiments, the compositions of the inventions comprise eriodictyol in a concentration of about 10 nM to about 4 mM. In certain embodiments, the compositions of the invention comprise eriodictyol in a concentration of about 5 nM to about 2500 nM. In certain embodiments, the compositions of the invention comprise eriodictyol in a concentration of about 10 nM to about 2000 nM. In certain embodiments, the compositions of the invention comprise eriodictyol in a concentration of about 20 nM to about 1000 nM. In certain embodiments, the compositions of the invention comprise eriodictyol in a concentration of about 10 nM to about 20 nM. In certain embodiments, the compositions of the inventions comprise eriodictyol in a concentration of about 5 μM to about 3,750 μM. In certain embodiments, the compositions of the inventions comprise eriodictyol in a concentration of about 50 μM to about 3,000 μM. In certain embodiments, the compositions of the inventions comprise eriodictyol in a concentration of about 50 μM to about 3,000 μM.

In certain embodiments, eriodictyol is present in a concentration of about 0.0001% to about 5.0% w/w in the compositions of the invention. In certain embodiments, eriodictyol is present in a concentration of about 0.0001% to about 1.0% w/w in the compositions of the invention. In certain embodiments, eriodictyol is present in a concentration of about 0.001% to about 0.5% w/w in the compositions of the invention. In certain embodiments, eriodictyol is present in a concentration of about 0.01% to about 0.5% w/w in the composition of the invention. In certain embodiments, eriodictyol is present in a concentration of about 0.1% to about 0.5% w/w in the compositions of the invention. In certain embodiments, eriodictyol is present in a concentration of about 0.5% w/w in the compositions of the invention.

In certain aspects, the compositions of the invention may be administered as a topical composition on the skin of the subject. The compositions of the invention may be included in any formulation suitable for topical administration. In certain embodiments, the composition of the invention is a fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, and/or hair treatment. In certain embodiments, the compositions of the invention also include excipients. These excipients may be selected from a group consisting of: solvent, emulsifier, preservative, antioxidant, emollient, thickening agent, penetration enhancer, surfactant, diluent, filler, carrier, and/or pH control agent. In certain embodiments, the preservative in the composition is an antimicrobial preservative or chelating agent.

The compositions of the invention may be prepared by various methods. As an example, the compositions may be prepared by dissolving, dispersing, etc. eriodictyol as provided herein and optional excipients in a carrier (e.g., water, saline, aqueous dextrose, glycerol, glycols, ethanol or the like) or a solvent to form a solution, colloid, liposome, emulsion, complexes (including inclusion complexes), coacervate, or suspension. In certain embodiments, the compositions of the current invention can also contain additional excipients such as wetting agents, emulsifying agents, co-solvents, solubilizing agents, pH buffering agents, and the like (e.g., sodium acetate, sodium citrate, cyclodextrins and derivatives, sorbitan monolaurate, triethanolamine acetate, triethanolamine oleate, hydroxyethylpiperazine and the like). In certain embodiments, the compositions of the invention may further include one or more additional excipients selected from a group consisting of: carriers, excipients, or diluents including, but not limited to, absorbents, anti-irritants, antibacterials, anti-acne agents, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

In certain embodiments, the compositions of the invention comprise: eriodictyol, in an oil/water emulsion, and a preservative.

In certain embodiments, the compositions of the invention comprise: eriodictyol, in an oil/water emulsion, a solvent for solubilizing eriodictyol, and a preservative.

In certain embodiments, the compositions of the invention comprise eriodictyol, in an oil/water emulsion, a solvent for solubilizing eriodictyol, a diluent, and a preservative.

In certain embodiments, the compositions of the invention comprise: eriodictyol, in an oil/water emulsion, a solvent for solubilizing eriodictyol, a thickener, optionally a diluent, and a preservative.

In certain embodiments, the compositions of the invention comprise: eriodictyol, in an oil/water emulsion, a solvent for solubilizing eriodictyol, and one or more excipients selected from the group consisting of: diluents, absorbents, antioxidants, emollients, emulsifiers, thickeners, surfactants, and/or preservatives.

In certain embodiments, the excipients included in the composition are water, dimethyl isosorbide, glycerin, caprylic/capric triglyceride, propanediol, sunflower seed oil, ethoxydiglycol, squalane, sodium acrylate copolymer, cetearyl alcohol, phenoxyethanol, glyceryl stearate, capryloyl glycerin/sebacic acid copolymer, diheptyl succinate, lecithin, decylene glycol, pentylene glycol, cetearyl glucoside, shea butter, palmitic acid, arachidyl alcohol, behenyl alcohol, arachidyl glucoside, phenethyl alcohol, 1,2-hexanediol, hydrogenated olive oil, Olea europaea (olive) fruit oil, Olea europaea (olive) oil unsaponifiables, hydroxyethyl acrylate/sodium acryloyldimethyl taurate copolymer, citric acid, sodium acetylated hyaluronate, sodium hyaluronate crosspolymer, sodium phytate, hydrolyzed sodium hyaluronate, and/or ethylhexylglycerin.

The compositions of the invention may be prepared by various methods. As an example, the compositions may be prepared by dissolving, dispersing, etc. eriodictyol as provided herein and optional excipients in a carrier (e.g., water, saline, aqueous dextrose, glycerol, glycols, ethanol or the like) or a solvent to form a solution, colloid, liposome, emulsion, complexes (including inclusion complexes), coacervate, or suspension. In certain embodiments, the compositions of the current invention can also contain auxiliary substances such as wetting agents, emulsifying agents, co-solvents, solubilizing agents, pH buffering agents, and the like (e.g., sodium acetate, sodium citrate, cyclodextrins and derivatives, sorbitan monolaurate, triethanolamine acetate, triethanolamine oleate, hydroxyethylpiperazine and the like). In certain embodiments, the compositions of the invention may further include one or more additional excipients selected from a group consisting of: carriers, excipients, or diluents including, but not limited to, absorbents, anti-irritants, antibacterials, anti-acne agents, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

Absorbents are substances which are added to topical products to take up water and oil-soluble dissolved or finely dispersed substances. Topical absorbents can also be used as thickeners in a wide variety of formulations including facial creams, lipsticks, shampoos and calamine lotions. In some embodiments, absorbents may include, but are not limited to, alcohol (ethyl alcohol, methanol, isopropyl alcohol, SD alcohol [especially denatured alcohol] and benzyl alcohol), alumina (aluminum oxide), aluminum chlorohydrate, aluminum hydroxide, aluminum magnesium silicate, aluminum silicate, aluminum starch octenylsuccinate, aluminum sulfate, ammonium chloride, bentonite, bismuth oxychloride, boron nitride, calcium carbonate, carnauba wax, charcoal, China clay, clay, Copernicia cerifera wax, cornstarch, fuller's earth, hydrolyzed corn starch, iron powder, kaolin, lithium magnesium sodium silicate, magnesium, magnesium carbonate, magnesium hydroxide, montmorillonite, nylon-12, rice starch, silica, silicate, silk powder, silt, sodium carbonate, sodium polyacrylates, and zeolite.

Anti-Irritants are substances which are added to topical products to reduce inflammation, especially, the inflammation resulting from the application of the product itself. The excipients included as anti-irritants that perform the function of anti-irritants or anti-inflammatories may have more than one function. For example, many antioxidants also function as anti-irritants. In some embodiments, anti-irritants and/or antioxidants may include, but are not limited to, Acacia senegal, acetylsalicylic acid, Achillea millefolium, adenosine, citric acid, adenosine triphosphate Aloe barbadensis, aloe barbadensis leaf juice extract, aloe extract, aloe juice, hydrogenated olive oil, hydrogenated palm glycerides, hydrolyzed silk, hydroquinone, aloe vera, and/or alpha bisabolol.

In certain embodiments, the compositions of the invention may further include humectants. Humectants (or moisturizers) are important cosmetic ingredients that prevent loss of moisture thereby retaining the skin's natural moisture. Some humectants also can actively attract moisture. In certain embodiments, the humectants may be selected from a group consisting of sodium hyaluronate, sodium acetylated hyaluronate, hydrolyzed sodium hyaluronate, propylene glycol, hexylene glycol, panthenol, pentylene glycol, and butylene glycol.

In certain embodiments, the compositions of the invention may further include slip agents. The term “slip agent” is used to describe a range of ingredients which can help other ingredients spread over the skin and penetrate into the skin. Slip agents can also have humectant (hygroscopic) properties. In some embodiments, slip agents may include, but are not limited to, amodimethicone, bis-PEG-18 methyl ether dimethyl silane, bis-phenylpropyl dimethicone, butylene glycol, cetyl dimethicone, cetyl dimethicone copolyol, cetyl PEG/PPG-10/1-dimethicone, cyclohexasiloxane, cyclomethicone, cyclopentasiloxane, cyclotetrasiloxane, decylene glycol, diisostearoyl trimethylolpropane siloxy silicate, dimethicone, dimethicone copolyol, dimethicone crosspolymer, dimethiconol, dipropylene glycol, hexylene glycol, hydrolyzed silk, isododecane, methicone, methyl trimethicone, methylsilanol mannuronate, methylsilanol PEG-7 glyceryl cocoate, PEG-10 dimethicone, PEG-10 dimethicone/vinyl dimethicone crosspolymer, PEG-12 dimethicone, PEG/PPG-18/18 dimethicone, PEG/PPG-20/15 dimethicone, pentylene glycol, phenyl trimethicone, polymethylsilsesquioxane, PPG-3 benzyl ether myristate, silica dimethyl silylate, silk powder, siloxane, simethicone, sorbitol, stearyl dimethicone, stearyl methicone, tricthoxycaprylylsilane, trimethylsiloxysilicate, xylitol, and zinc stearate.

In certain embodiments, the compositions of the invention may further include diluents, and/or carriers. Diluents and/or carriers may include, but are not limited to, polyethylene glycols (such as PEG200, PEG300, PEG400, PEG540, PEG600, PEG1450 or mixtures thereof) and coconut oils (such as propylene glycol dicaprate, coco-caprylate/caprate, propylene glycol dicaprylate/dicaprate, caprylic/capric triglyceride, caprylic/capric/lauric triglyceride, caprylic/capric/linoleic triglyceride, tricaprin, tricaprylin, glyceryl trioleate, neopentyl glycol dicaprylate/dicaprate, caprylic/capric/palmitic/stearic trigylceride, or mixtures thereof).

In certain embodiments, the compositions of the invention may further include emollients. Emollients are supple, waxlike, lubricating, thickening substances which are added to cosmetic/dermatological products to prevent water loss and have a softening and soothing effect on the skin. In some embodiments, emollients may include, but are not limited to, 10-hydroxydecanoic acid, acetyl glyceryl ricinoleate, acetylated castor oil, acetylated hydrogenated cottonseed glyceride, acetylated lanolin, acetylated lanolin alcohol, acetylated palm kernel glycerides, agar, algae extract, algin, almond oil, squalane, squalene, α-glucan oligosaccharide, amodimethicone, Anacystis nidulans extract, apricot kernel oil, arachidic acid, arachidonic acid, arachidyl alcohol, arachidyl propionate, C₁₀₋₃₀ cholesterol/lanosterol esters, C₁₂₋₁₅ alkyl benzoate, C₁₂₋₁₈ acid triglyceride, C₁₈₋₃₆ acid triglyceride, candelilla wax, Cannabis sativa L. oil, caprylic/capric triglyceride, caprylyl methicone, carrot oil, Carthamus tinctorius oil, Carya illinoensis oil, castor isostearate succinate, castor oil, Caulerpa taxifolia extract, and/or cephalin.

In certain embodiments, the compositions of the invention may further include film-forming/holding agents. Film-Forming/Holding agents are substances which are added to cosmetic/dermatological products to help leave a pliable, cohesive, and continuous covering over the skin. This film has water-binding properties and leaves a smooth feel on skin. In some embodiments, film-forming/holding agents may include, but are not limited to, acrylate, acrylates copolymer, acrylates/C₁₀₋₃₀ alkyl acrylate crosspolymer, acrylates/dimethicone copolymer, adipic acid/neopentyl glycol/trimellitic anhydride copolymer, allyl methacrylates crosspolymer, carnauba wax, cellulose gum, Copernicia cerifera wax, dextrin, diisopropyl adipate, glyceryl polymethacrylate, hydrolyzed wheat protein, hydroxyethylcellulose, locust bean, neopentyl glycol diheptanoate, PEG-40 hydrogenated castor oil, polyacrylamide, polyacrylate-17, polyglucuronic acid, polyglyceryl methacrylate, polyisobutene, polymethyl methacrylate, polyquaternium-10, polyquaterniums, polyvinyl alcohol, polyvinylpyrrolidone, propylene carbonate, PVM/MA (polyvinyl methyl ether/maleic acid) decadiene crosspolymer, PVP [poly(vinylpyrrolidone)] copolymer, PVP/dimethylaminoethyl-methacrylate, sodium carbomer, sodium polyacrylate, styrene/acrylates copolymer, triethoxycaprylylsilane crosspolymer, VA (vinyl acetate)/crotonates, VA/crotonates copolymer, VP (vinylpyrrolidone)/eicosene copolymer, and VP/hexadecene copolymer.

In certain embodiments, the compositions of the invention may further include excipients related to the fragrance of the formulation. Fragrance ingredients are a single or a blend of volatile and/or fragrant plant oils (or synthetically derived oils) that impart aroma and odor to cosmetic/dermatological products. The level of fragrance to use varies according to the product type. In some embodiments, the composition of the formulation may contain up to about 0.01% fragrance by weight. In some embodiments, fragrances (e.g., synthetic and fragrant plant extracts) may include, but are not limited to, Acacia farnesiana extract, Aerocarpus santalinus, amyl cinnamate, amyl salicylate, amyris oil, Anethum graveolens, Angelica archangelica root oil, anisaldehyde, anise, balm mint extract, balsam peru, bay leaf oil, bergamot oil, bitter orange flower, bois de rose oil, bois oil, Boswellia carterii, butylphenyl methylpropional, cananga extract, Cananga odorata, cardamom, cedarwood, cherry extract, Citrus aurantifolia, Citrus aurantium, Citrus aurantium extract, Citrus medica limonium, clary oil, Commiphora myrrha extract, coriander, Cucurbitea peponis, cyclamen aldehyde, dill extract, ethyl vanillin, eugenol, farnesol, farnesyl acetate, Ferula galbaniflua, fir needle oil, floralozone, Foeniculum vulgare extract, frankencense extract, galbanum, Gardenia florida extract, grapefruit oil, guaiac wood, Guaiacum officinale, hedione, hexyl cinnamal, hyssop, Illicium vernum, Iris florentina extract, jasmine oil, Jasminium grandiflorum, jonquil extract, Laurus nobilis, lauroyl lactate, lavandin oil, Lavandula angustifolia, Lavandula officinalis, lavender extract and oil, lemon, lemon balm, lemongrass oil, Levisticum officinale root extract, lime oil and extract, limonene, linalool, Litsea cubeba, mandarin orange oil or extract, marjoram, Melissa officinalis, Mentha piperita, Mentha spicata, Mentha viridis, menthol, menthone, menthoxypropanediol, and menthyl lactate.

In certain embodiments, the composition of the invention may further include preservatives. Preservatives are substances which prevent bacterial, microbial or fungal contamination of cosmetic/dermatological products thereby increasing the product's shelf life and consumer safety. Some of these agents also have stabilizing effects able to preserve the function of various active ingredients including anti-oxidants (vitamins), emulsifiers and surfactants. In some embodiments, preservatives may include, but are not limited to, 1,2-hexanediol, benzoic acid, benzothonium chloride, borax, bronopol, butylparaben, caprylyl glycol, chlorophene, chloroxylenol, chlorphenesin, decylene glycol, dehydroacetic acid, diazolidinyl urea, DMDM hydantoin, ethylhexylglycerin, ethylparaben, formaldehyde-releasing preservative, Germaben II, hoelen, hydroxyacetophenone, imidazolidinyl urea, iodopropynyl butylcarbamate, isobutylparaben, methylchloroisothiazolinone, methyldibromo glutaronitrile, Methylisothiazolinone, methylparaben, o-cymen-5-ol, phenoxyethanol, phenoxyisopropanol, phytosphingosine, polyaminopropyl biguanide, potassium sorbate, propylparaben, quaternium-15, sodium benzoate, sodium citrate, sodium dehydroacetate, sodium hexametaphosphate, sodium hydroxymethylglycinate, phenethyl alcohol, sodium lactobionate, sodium metabisulfite, sodium sulfite, sorbic acid, and styrax benzoin.

In certain aspects of the invention, the pharmaceutical composition is an emulsion. The emulsion compositions of the invention may further include emulsifiers. Emulsifiers are substances which are added to cosmetic/dermatological products to help keep unlike ingredients (such as oil and water) from separating in an emulsion. There are 2 types of emulsifiers. Oil-in-water (o/w) emulsifiers keep oil drops packed in water, while water-in-oil (w/o) emulsifiers keep water drops packed in oil. W/O emulsifiers are used for a fatty feel (e.g. night & sun protection creams). O/W emulsifiers are used more in moisturizing products (e.g. body lotions, day creams).

In certain embodiments, the compositions of the invention may further include surfactants. Surfactants are compounds that lower surface tension or interfacial tension. Reducing the surface tension allows a liquid to spread easier while reducing the interfacial tension (interfacial meaning between two faces) between water and an oil/lipid allows them to mix. Surfactants may have a be anionic, amphoteric, non-ionic, and/or quaternary agents. Surfactants form the base of all personal cleansing products and can also have wetting, conditioning, defatting, emulsifying, & thickening effects. In some embodiments, emulsifiers, surfactants, and detergents may include, but are not limited to, ammonium laureth sulfate, ammonium lauroyl sulfate, arachidyl glucoside, behenic acid, bis-PEG-18 methyl ether dimethyl silane, C₂₀₋₄₀ pareth-40, cocamidopropyl betaine, cocamidopropyl dimethylamine, cocamidopropyl hydroxysultaine, coco-glucoside, coconut oil, decyl glucoside, dicetyl phosphate, dihydrocholeth-30, disodium cocoamphodiacetate, disodium cocoyl glutamate, disodium lauraminopropionate, glyceryl behanate, hydrogenated vegetable glycerides citrate, isohexadecane, isostearamide DEA, lauramphocarboxyglycinate, laureth-23, laureth-4, laureth-7, lauroyl PEG-9 polydimethylsiloxyethyl dimethicone, lauroyl alcohol, lauroyl glucoside, magnesium laureth sulfate, magnesium oleth sulfate, myristic acid, nonoxynols, oleic acid, oleth 10, palm kernel acid, palmitic acid, PEG-60 almond glycerides, PEG-75 shea butter glycerides, PEG 90M, PEG-10 dimethicone, PEG-10 dimethicone/vinyl dimethicone crosspolymer, PEG-10 rapeseed sterol, PEG-100 stearate, PEG-12 dimethicone, PEG-120 methyl glucose dioleate, PEG-20 methyl glucose sesquistearate, PEG-40 stearate, PEG-60 hydrogenated castor oil, PEG-7 glyceryl cocoate, PEG-8, PEG-80 sorbitan laurate, PEG/PPG-17/6 copolymer (polyethylene glycol/polypropylene glycol-17/6 copolymer), PEG/PPG-18/18 dimethicone, PEG/PPG-20/15 dimethicone, poloxamer 184, Poloxamer 407, poloxamers, polyglyceryl-3 beeswax, polyglyceryl-4 isostearate, polyglyceryl-6 isostearate, polysorbate 20, polysorbate 60, polysorbate 80, potassium cetyl phosphate, potassium hydroxide, potassium myristate, PPG-12 buteth-16, PPG-26-Buteth-26, Salvia officinalis, Saponaria officinalis extract, soapwort, sodium C₁₄₋₁₆ olefin sulfonate, sodium cetearyl sulfate, sodium cocoamphoacetate, sodium cocoate, sodium cocoyl glutamate, sodium cocoyl isethionate, sodium dilauramidoglutamide lysine, sodium hexametaphosphate, sodium hydroxide, sodium laureth sulfate, sodium laureth-13 carboxylate, sodium lauroamphoacetate, sodium lauroyl lactylate, sodium lauroyl sarcosinate, sodium lauroyl glucose carboxylate, sodium lauroyl sulfate, sodium methyl cocoyl taurate, sodium methyl taurate, sodium myreth sulfate, sodium palm kernelate, sodium palmate, sodium PEG-7 olive oil carboxylate, sodium trideceth sulfate, steareth-20, TEA-lauroyl sulfate (triethanolamine-lauroyl sulfate), and tribehenin PEG-20 esters.

In certain embodiments, the compositions of the invention may further include one or more thickeners. Thickeners are substances which are added to cosmetic/dermatological products to enhance the consistency, volume and viscosity of cosmetic products, thereby providing more stability and better performance. While some thickeners have also emulsifying or gelling properties, the majority of thickeners have the ability to retain water on the skin and act therefore as moisturizers. Thickeners can be completely natural like waxes but also synthetic or semi-synthetic. In some embodiments, thickeners may include, but are not limited to, Acacia senegal, acetyl glyceryl ricinoleate, acetylated castor oil, acetylated hydrogenated cottonseed glyceride, acetylated palm kernel glycerides, acrylates/steareth-20 methacrylate copolymer, agar, Ahnfeltia concinna extract, ahnfeltia extract, Alaria esculenta, algae, algae extract, algin, alkyloamides, Althaea rosea, Althea officinalis, alumina, aluminum hydroxide, aluminum stearate, ammonium acryloyldimethyltaurate/VP copolymer, Anacystis nidulans extract, arachidic acid, arachidonic acid, arachidyl alcohol, arachidyl propionate, arrowroot, artemia extract, Ascophyllum nodosum, ascorbyl methylsilanol pectinate, Asparagopsis armata extract, beeswax, behenic acid, behentrimonium chloride, behenyl alcohol, bis-diglyceryl polyacyladipate, bismuth oxychloride, C₁₂₋₁₅ alkyl benzoate, C₁₂₋₁₈ acid triglyceride, C₁₃₋₁₄ isoparaffin, C₁₈₋₃₆ acid triglyceride, candelilla wax, caprylic/capric triglyceride, carbomer, carbopol, carnauba wax, carrageenan, Caulerpa taxifolia extract, cellulose, Cellulose gum, cephalin, Cera alba, ceresin, ceteareth-20, cetearyl alcohol, cetearyl ethylhexanoate, cetearyl glucoside, cetearyl octanoate, cetyl alcohol, cetyl dimethicone copolyol, cetyl hydroxyethylcellulose, cetyl palmitate, cetyl PEG/PPG-10/1-dimethicone, chlorella, Chondrus crispus, cocamide DEA and MEA, cocoglycerides, Codium tomentosum extract, Copernicia cerifera wax, Corallina officinalis extract, Corallina, DEA oleth-10 phosphate, diethanolamine (DEA), di-PPG-3 myristyl ether adipate, dimethicone crosspolymer, dimethicone/vinyl dimethicone crosspolymer, polyacrylate crosspolymer, ammonium 2 acrylamido-2-methylpropane sulfonic acid crosspolymer, sodium polyacryloyldimethyl taurate, tara gum, and/or dimer dilinoleyl dimer dilinoleate.

In certain embodiments, the compositions of the invention may further include one or more chelators. Chelating agents are any of numerous ingredients that bind with metal ions or metallic compounds, preventing them from adhering to a surface (such as skin, hair, or clothing) or causing contamination, such as in the case of trace amounts of iron. In some embodiments, chelators may include, but are not limited to tetrasodium EDTA, sodium phytate, and/or teathydroxypropyl ethylenediamine.

In certain embodiments, the compositions of the invention include one or more buffers and/or pH-adjustors. The buffer and/or pH adjustor (also referred to as pH control agent) is present in the compositions of the invention to maintain the pH of the solution in an optimal range. In certain embodiments, the one or more buffer and/or pH adjustors are selected from a group consisting of: phosphoric acid/phosphate, citric acid/citrate, tromethamine, histidine, lactic acid, gluconic acid, aspartic acid, glutamic acid, tartaric acid, glutamic acid, succinic acid, malic acid, fumaric acid, and/or α-ketoglutaric.

In certain embodiments, the excipients included in the composition are water, dimethyl isosorbide, glycerin, caprylic/capric triglyceride, propanediol, sunflower seed oil, ethoxydiglycol, squalane, sodium acrylate copolymer, cetearyl alcohol, phenoxyethanol, glyceryl stearate, capryloyl glycerin/sebacic acid copolymer, diheptyl succinate, naringenin, lecithin, decylene glycol, pentylene glycol, cetearyl glucoside, shea butter, palmitic acid, arachidyl alcohol, behenyl alcohol, arachidyl glucoside, phenethyl alcohol, 1,2-hexanediol, hydrogenated olive oil, Olea europaea (olive) fruit oil, Olea europaea (olive) oil unsponifiables, hydroxyethyl acrylate/sodium acryloyldimethyl taurate copolymer, citric acid, sodium acetylated hyaluronate, sodium hyaluronate crosspolymer, sodium phytate, hydrolyzed sodium hyaluronate, and/or ethylhexylglycerin.

Table 3 provides a list of excipients along with their approximate concentrations, that may be used in the exemplary compositions of the invention.

TABLE 3
Exemplary excipients along with their concentration ranges:

| | Concentration |
|---|---|
| Ingredient | % w/w |
| Water | 30-90 |
| Glycerin | 0-20 |
| Propanediol | 0-10 |
| Hydrogenated Ethylhexyl Olivate | 0-2 |
| Hydrogenated Olive Oil Unsaponifiables | 0-2 |
| Polyglyceryl-6 Distearate | 0-5 |
| Jojoba Esters | 0-2.5 |
| Polyglyceryl-3 Beeswax | 0-2 |
| Cetyl Alcohol | 0-2 |
| Cetearyl Olivate | 0-5 |
| Sorbitan Olivate | 0-5 |
| Hydroxyethyl Acrylate/Sodium Acryloyldimethyl Taurate | 0-5 |
| Copolymer | |
| Helianthus Annuus (Sunflower) Seed Oil | 0-10 |
| Caprylic/Capric Triglyceride | 0-10 |
| Phenoxyethanol | 0-2 |
| Decylene Glycol | 0-2 |
| 1,2-Hexanediol | 0-2 |
| Eriodictyol | 0.0001-5 |
| Dimethyl Isosorbide | 0-10 |

### Eriodictyol for Treatment of Topical Conditions:

Advantageously, it was discovered that eriodictyol impacts the genes and signaling pathways involved in several topical conditions. In one aspect, the invention provides methods of using the compositions for treating, protecting, and/or altering (e.g., improving) the condition and/or aesthetic appearance of skin, including, for example, treating, preventing, ameliorating, reducing and/or eliminating fine lines and/or wrinkles of skin and/or improving the aesthetic appearance of fine lines and/or wrinkles of skin. In certain embodiments, the compositions of the invention lead to increase in skin and/or hair resiliency. In certain embodiments, the compositions of the invention lead to increase in elasticity of the skin. In certain embodiments, the compositions of the invention prevent skin damage.

In one aspect, the invention includes a composition for topical administration comprising an effective amount of eriodictyol. The effective amount of eriodictyol is an amount of eriodictyol that would be effective in treating, ameliorating, or otherwise impacting the underlying condition of the skin.

In certain aspects of the invention, the compositions comprising eriodictyol demonstrate antioxidant activity. In certain embodiments, the compositions of the invention, the antioxidant activity of eriodictyol is demonstrated at concentrations of about 0.00016% w/w or higher. Beneficially, the antioxidant activity of compositions comprising eriodictyol is demonstrated in both aqueous and organic solvent systems. Thus, the invention provides compositions which retain antioxidant activity in both aqueous and organic solvents. Advantageously, the compositions of the invention comprising eriodictyol, demonstrating antioxidant activity, are effective for treating and/or preventing oxidation damage. In certain embodiments, the compositions of the invention comprising eriodictyol, demonstrating antioxidant activity, are effective for treatment of skin and/or hair damaged by reactive oxygen species and protection from other environmental aggressors that may damage the skin of the subject. In certain other embodiments, the compositions of the invention comprising eriodictyol, demonstrating antioxidant activity, are effective for prevention of skin and/or hair damage by reactive oxygen species, including the damage caused by environmental aggressors.

As demonstrated from the data provided in Examples 2 and 3, compositions comprising eriodictyol are effective in demonstrating robust antioxidant activity in both aqueous and organic solvent systems. The IC₅₀ values for antioxidant activity calculated in aqueous solvent system was 0.00062% w/w. The IC₅₀ values for antioxidant activity calculated in organic solvent system was 0.0002% w/w. These results demonstrate that compositions comprising eriodictyol have robust antioxidant activity. In certain embodiments, the compositions comprising eriodictyol are useful in treatment of conditions caused by oxidation or excessive oxidation in the skin of the subject. In certain embodiments, the compositions comprising eriodictyol are administered for treatment and/or prevention of skin or hair damage by reaction oxygen species and protection from environmental aggressors.

In certain aspects of the invention, the compositions comprising eriodictyol demonstrate elastase enzyme inhibition. In certain embodiments, the compositions of the invention, the elastase enzyme inhibition activity of eriodictyol is demonstrated at concentrations of about 0.008% w/w or higher. The data for elastase enzyme inhibition for the treatment for enzyme inhibition is provided in Example 4. The IC₅₀ values for elastase enzyme inhibition by compositions comprising eriodictyol is 0.107% w/w. Advantageously, the compositions comprising eriodictyol, demonstrating elastase enzyme inhibition activity, are effective for promoting the elasticity of the skin. In certain embodiments, the compositions of the invention comprising eriodictyol, demonstrating elastase enzyme inhibition, are effective in reduction of fine lines and/or wrinkles in the skin. In certain embodiments, the compositions of the invention comprising eriodictyol, demonstrating elastase enzyme inhibition, are effective in prevention of formation of fine lines and/or wrinkles in the skin.

In certain aspects of the invention, the compositions comprising eriodictyol demonstrate inhibition of glycation. In certain embodiments, the compositions of the invention, the glycation inhibition activity of eriodictyol is demonstrated at concentrations of about 0.002% w/w or higher. The data for elastase enzyme inhibition for the treatment of glycation inhibition is provided in Example 5. The IC₅₀ values for glycation inhibition by compositions comprising eriodictyol is 0.0088% w/w. Advantageously, the compositions comprising eriodictyol, demonstrating glycation inhibition activity, are effective for supporting and promoting skin and/or hair resiliency.

Gene Expression and/or Activity Modulation

In certain embodiments of the invention, the compositions of the invention, comprising eriodictyol modulate the gene expression of one or more genes that is related to a topical condition. In certain embodiments, administration of composition comprising eriodictyol alters one or more of a function related with the modulated genes, wherein the one or more function is selected from the group consisting of: (i) formation of fines lines or wrinkles on the skin, (ii) inflammation, (iii) skin elasticity, (iv) prevention of scar formation during wound healing, and/or (v) any combination thereof.

In certain embodiments, administration of composition comprising eriodictyol alters expression and/or function of one or more genes are selected from a group consisting of: MMP10, THBS1, TOP2A, TFPI2, MMP1, FN1, KCTD4, ATP12A, ALDH1A3, MKI67, FST, SLC13A5, IL6, IL23A, TNF, IL24, MMP3, MMP9, and/or PPFIA4. Example 6 (Table 9) provides an overview of the gene modulation by compositions comprising eriodictyol.

In certain embodiments, administration of composition comprising eriodictyol leads to down-regulation of expression and/or activity of MMP10, THBS1, TOP2A, TFPI2, MMP1, FN1, KCTD4, ATP12A, ALDH1A3, MKI67, FST, SLC13A5, IL6, IL23A, TNF, IL24, MMP3, and/or MMP9.

In certain embodiments, the administration of the composition leads to up-regulation of expression and/or activity of PPFIA4.

In certain embodiments, the administration of the composition enhances anti-inflammatory activity. In certain embodiments, the anti-inflammatory activity of the composition is modulated through the downregulation of expression and/or activity of IL6, IL23a, TNF, and/or IL24.

In certain embodiments, the administration of the composition leads to anti-aging activity. In certain embodiments, the anti-aging activity of the composition comprising eriodictyol is modulated through downregulation of expression and/or activity of MMP10, MMP9, MMP3, and MMP1.

In certain embodiments, the administration of the composition comprising eriodictyol enhances maintenance of skin barrier lipids. In certain embodiments, the maintenance of skin barrier lipids is modulated by upregulation of expression and/or activity of PPFIA4.

In certain embodiments, the administration of the composition comprising eriodictyol results in prevention of formation of scar tissue after healing of wounds. In certain embodiments, the prevention of formation of scar tissue after healing of wounds is modulated by downregulation of expression and/or activity of THBS1.

In certain embodiments, the administration of the composition comprising eriodictyol leads to at least about ten (10) fold downregulation of the activity and/or expression of matrix metallopeptidase 10 (MMP10). In certain embodiments, administration of the composition comprising eriodictyol leads to about twenty-five (25) fold downregulation of the activity and/or expression of matrix metallopeptidase 10 (MMP10).

Thrombospondin 1 (THBS1) plays a role in proliferation of hypertrophic scar cells. In certain embodiments, hypertrophic scar cells play a role in formation of scars in wound healing. Thus, downregulation of THBS1 leads to prevention of scar formation upon wound healing. In certain embodiments, administration of the composition comprising eriodictyol leads to at least about ten (10) fold downregulation of the activity and/or expression of thrombospondin 1 (THBS1). In certain embodiments, administration of the composition comprising eriodictyol leads to about fifteen (15) fold downregulation of the activity and/or expression of thrombospondin 1 (THBS1).

DNA topoisomerase II-α (TOP2A) expresses an enzyme that is elevated in expression in tumors. It is an enzyme that controls and alters the topologic states of DNA during transcription. In certain embodiments, administration of the composition comprising eriodictyol leads to at least about ten (10) fold downregulation of the activity and/or expression of DNA topoisomerase II-α (TOP2A). In certain embodiments, administration of the composition comprising eriodictyol leads to about fifteen (15) fold downregulation of the activity and/or expression of DNA topoisomerase II-α (TOP2A). In certain embodiments, administration of the composition comprising eriodictyol leads to about twenty (20) fold downregulation of the activity and/or expression of DNA topoisomerase II-α (TOP2A).

Tissue factor pathway inhibitor 2 (TFPI2) expresses an antimicrobial protein during wound healing. In certain embodiments, downregulation of activity and/or expression of TFPI2 may assist in wound healing and/or prevention of formation of scars in the process of wound healing. In certain embodiments, wherein the administration of the composition comprising eriodictyol leads to at least about ten (10) fold downregulation of the activity and/or expression of tissue factor pathway inhibitor 2 (TFPI2). In certain embodiments, administration of the composition comprising eriodictyol leads to about fifteen (15) fold downregulation of the activity and/or expression of tissue factor pathway inhibitor 2 (TFPI2). In certain embodiments, administration of the composition comprising eriodictyol leads to about twenty (20) fold downregulation of the activity and/or expression of tissue factor pathway inhibitor 2 (TFPI2).

Matrix metallopeptidase 1 (MMP1) expresses an enzyme that may be responsible for appearance of aging in the skin. UV irradiation induces increased synthesis and expression of MMP-1 by dermal fibroblasts, which is stimulated by the generation of excess reactive oxygen species (ROS), and plays a critical role in photoaging. Thus, downregulation of expression and/or activity of MMP-1 may lead to an anti-aging effect on the skin. In certain embodiments, administration of the composition comprising eriodictyol leads to at least about two (2) fold downregulation of the activity and/or expression of matrix metallopeptidase 1 (MMP1). In certain embodiments, administration of the composition comprising eriodictyol leads to about ten (10) fold downregulation of the activity and/or expression of matrix metallopeptidase 1 (MMP1).

Fibronectin 1 (FN1) expresses a protein that is associated with wound healing. Increased expression of FN1 is associated with wound healing. In certain embodiments, downregulation of expression and/or activity of FN1 may be associated with reduction of formation of scars during the process of wound healing. In certain embodiments, administration of the composition leads to at least about two (2) fold downregulation of the activity and/or expression of fibronectin 1 (FN1). In certain embodiments, administration of the composition leads to about ten (10) fold downregulation of the activity and/or expression of fibronectin 1 (FN1).

Potassium channel tetramerization domain containing 4 (KCTD4) expresses a protein that may be involved in protein homooligomerization. In certain embodiments, administration of the composition leads to at least about five (5) fold downregulation of the activity and/or expression of potassium channel tetramerization domain containing 4 (KCTD4). In certain embodiments, administration of the composition leads to about fifteen (15) fold downregulation of the activity and/or expression of potassium channel tetramerization domain containing 4 (KCTD4).

ATPase H+/K+ transporting non-gastric α2 subunit (ATP12A) encodes a catalytic subunit of the ouabain-sensitive H+/K+-ATPase that catalyzes the hydrolysis of ATP coupled with the exchange of H(+) and K(+) ions across the plasma membrane. It is also responsible for potassium absorption in various tissues. In certain embodiments, administration of the composition comprising eriodictyol leads to at least about two (2) fold downregulation of the activity and/or expression of ATPase H+/K+ transporting non-gastric α2 subunit (ATP12A). In certain embodiments, administration of the composition comprising eriodictyol leads to about ten (10) fold downregulation of the activity and/or expression of ATPase H+/K+ transporting non-gastric α2 subunit (ATP12A).

Aldehyde dehydrogenase 1 family member A3 (ALDH1A3) expresses an enzyme that converts retinaldehyde to retinoic acid. Upregulation of ALDH1A3 is associated with tumor formation, and downregulation indicates reduced cell proliferation. In certain embodiments, administration of the composition comprising eriodictyol leads to at least about two (2) fold downregulation of the activity and/or expression of aldehyde dehydrogenase 1 family member A3 (ALDH1A3). In certain embodiments, administration of the composition comprising eriodictyol leads to about ten (10) fold downregulation of the activity and/or expression of aldehyde dehydrogenase 1 family member A3 (ALDH1A3).

Marker of proliferation Ki-67 (MKI67) expresses a protein involved in cell proliferation. Downregulation of expression and/or activity of MKI67 is associated with decreased cell proliferation. In certain embodiments, administration of the composition comprising eriodictyol leads to at least about two (2) fold downregulation of the activity and/or expression of marker of proliferation Ki-67 (MKI67). In certain embodiments, administration of the composition comprising eriodictyol leads to about ten (10) fold downregulation of the activity and/or expression of marker of proliferation Ki-67 (MKI67).

Follistatin (FST) expresses a single chain glycoprotein structurally unrelated to inhibin and activin. Follistatin binds activins with high affinity and antagonizes the effects of activins by blocking the binding of activins to their receptors. Downregulation of activity and/or expression of FST results in enhanced keratinocyte proliferation. In certain embodiments, administration of the composition comprising eriodictyol leads to at least about two (2) fold downregulation of the activity and/or expression of follistatin (FST). In certain embodiments, administration of the composition comprising eriodictyol leads to about ten (10) fold downregulation of the activity and/or expression of follistatin (FST).

Solute carrier family 13 member 5 (SLC13A5) expresses a protein sodium-dependent citrate cotransporter that may regulate metabolic processes. Downregulation of activity and/or expression of SLC13A5 is associated with reduced risk of diabetes and increased lifespan. In certain embodiments, the activity of protein expressed by SLC13A5 may be affected by activity of interleukin-6. In certain embodiments, administration of the composition comprising eriodictyol leads to at least about five (5) fold downregulation of the activity and/or expression of solute carrier family 13 member 5 (SLC13A5). In certain embodiments, administration of the composition comprising eriodictyol leads to about fifteen (15) fold downregulation of the activity and/or expression of solute carrier family 13 member 5 (SLC13A5).

Interleukin 6 (IL6) expresses a protein involved in the inflammatory response in the body. Thus, downregulation of activity and/or expression of IL6 results in reduction of inflammatory responses in the skin. In certain embodiments, administration of the composition comprising eriodictyol leads to at least about five (5) fold downregulation of the activity and/or expression of interleukin 6 (IL6). In certain embodiments, administration of the composition comprising eriodictyol leads to about twenty (20) fold downregulation of the activity and/or expression of interleukin 6 (IL6).

Interleukin 23 (IL23) expresses a protein involved in the inflammatory response in the body. Thus, downregulation of activity and/or expression of IL23 results in reduction of inflammatory responses in the skin. In certain embodiments, administration of the composition comprising eriodictyol leads to at least about two (2) fold downregulation of the activity and/or expression of interleukin 23 (IL23). In certain embodiments, administration of the composition comprising eriodictyol leads to about five (5) fold downregulation of the activity and/or expression of interleukin 23 (IL23).

Tumor necrosis factor (TNF) expresses a protein involved in the inflammatory response in the body. Thus, downregulation of activity and/or expression of TNF results in reduction of inflammatory responses in the skin. In certain embodiments, administration of the composition comprising eriodictyol leads to at least about ten (10) fold downregulation of the activity and/or expression of tumor necrosis factor (TNF). In certain embodiments, administration of the composition comprising eriodictyol leads to about two hundred fifty (250) fold downregulation of the activity and/or expression of tumor necrosis factor (TNF).

Interleukin 24 (IL24) expresses a protein involved in the inflammatory response in the body. Thus, downregulation of activity and/or expression of IL24 results in reduction of inflammatory responses in the skin. In certain embodiments, administration of the composition comprising eriodictyol leads to at least about ten (10) fold downregulation of the activity and/or expression of interleukin 24 (IL24). In certain embodiments, administration of the composition comprising eriodictyol leads to about seventy (70) fold downregulation of the activity and/or expression of interleukin 24 (IL24).

Matrix metallopeptidase 3 (MMP3) expresses a protein involved in several signaling pathways, including collagen degradation and inflammation. Thus, downregulation of activity and/or expression of activity of MMP3 results in anti-aging effect and/or anti-inflammatory responses in the skin. In certain embodiments, administration of the composition comprising eriodictyol leads to at least about ten (10) fold downregulation of the activity and/or expression of matrix metallopeptidase 3 (MMP3). In certain embodiments, administration of the composition comprising eriodictyol leads to about fifty (50) fold downregulation of the activity and/or expression of matrix metallopeptidase 3 (MMP3).

Matrix metallopeptidase 9 (MMP9) expresses a protein involved in several signaling pathways, including collagen degradation and inflammation. Thus, downregulation of activity and/or expression of activity of MMP9 results in anti-aging effect and/or anti-inflammatory responses in the skin. In certain embodiments, administration of the composition comprising eriodictyol leads to at least about two (2) fold downregulation of the activity and/or expression of matrix metallopeptidase 9 (MMP9). In certain embodiments, administration of the composition comprising eriodictyol leads to about eight (8) fold downregulation of the activity and/or expression of matrix metallopeptidase 9 (MMP9).

PTPRF interacting protein α4 (PPFIA4) expresses a protein that plays a role in cell-adhesion. Thus, increased expression of PPFIA4 results in increased barrier function. In certain embodiments, administration of the composition comprising eriodictyol leads to at least about five (5) fold upregulation of the activity and/or expression of stimulated by PTPRF interacting protein α4 (PPFIA4). In certain embodiments, administration of the composition comprising eriodictyol leads to about thirty (30) fold upregulation of the activity and/or expression of PTPRF interacting protein α4 (PPFIA4).

### Treatment of Topical Conditions

In certain aspects, the invention provides methods of treatment of a topical condition by administration of the topical composition comprising an effective amount of eriodictyol. In certain embodiments, the invention provides methods for prevention of fine lines or wrinkles on skin of the subject by administration of the compositions provided herein. In certain embodiments, the invention provides methods for supporting firmness and increasing/maintaining elasticity of skin by administration of the compositions provided herein. In certain embodiments, the invention provides methods for prevention of skin and hair damaged by reactive oxygen species by administration of the compositions provided herein. In certain embodiments, the invention provides methods for protection from environment aggressors on skin by administration of the compositions provided herein. In certain embodiments, the invention provides methods for increase in skin and/or hair resiliency by administration of the compositions provided herein. In certain embodiments, the invention provides methods for prevention of glycation by administration of the compositions provided herein. Glycation is known to be linked with the wrinkling, loss of elasticity, and aging in the skin. In certain embodiments, the invention provides methods of reducing damage in skin by administration of compositions provided herein. In certain embodiments, the compositions of the invention provide methods for prevention of scar formation during the process of wound healing by administration of the compositions provided herein.

The current invention recognizes that the compositions comprising eriodictyol is beneficial for treating, protecting and/or improving the condition and/or aesthetic appearance of skin. For example, the compositions of the invention may be effective for altering the aesthetic appearance of skin associated with or affected by, or for treating or preventing, fine lines and/or wrinkles of skin caused by, for example, cellular senescence, environmental damage or dermatoheliosis. The invention also provides methods for stimulating skin cell renewal, increasing cell or tissue regeneration, promoting fibroblast proliferation and synthesizing elastin, collagen, proteoglycans, and/or new connective tissue, thereby reducing or improving the appearance of wrinkles, restoring elasticity, resiliency, and/or suppleness to the skin.

The compositions of the invention provided herein can be useful for improving the aesthetic appearance of skin. Such improvements can include, but are not limited to, all outward visibly and tactilely perceptible manifestations as well as any other macro or micro effects due to skin aging and damage. Such manifestations and effects can be induced or caused by intrinsic factors and/or extrinsic factors, e.g., chronological aging, environmental damage, climate, sun (UV) exposure, smoking, drugs, alcohol consumption, jetlag, night work, changes in circadian rhythm, pregnancy, menopause, genetic factors, nutritional factors and/or deficiencies, dehydration, stress, allergies (e.g., to plants, animals, medications, and other substances), exposure to industrial and/or household chemicals, indoor heating and cooling, various disorders and diseases such as arteriosclerosis, diabetes, heart disease, liver disease, and obesity, thinning of the outer layer of skin, decreases in the number of pigment-containing cells, increases in the size of pigment-containing cells, changes in the connective tissue, and reduction in the strength and elasticity of the skin.

The aesthetic appearance of skin can be improved, for example, by improving the appearance of skin associated with or affected by one or more of wrinkles, dry skin, sensitive skin; wrinkling and sagging; acne; vitiligo (skin condition in which there is a loss of brown color (pigment) from areas of skin); fine lines, wizened skin, thinning of the dermis, the degradation of collagen fibers, flaccid skin, thinned skin, and skin exposed to ultraviolet radiation. In some embodiments, the compositions of the invention can improve the aesthetic appearance of skin by decreasing the appearance of fine lines in the skin; creating a more youthful appearance of the skin; decreasing bags and/or rings around the eyes; increasing or restoring the elasticity, resiliency, and/or suppleness of the skin; increasing the apparent thickness, elasticity, flexibility, radiance, glow, and plumpness of the skin; improving the fineness of the skin texture; improving the appearance of wrinkles, lined, dry, flaky, aged, and/or photodamaged skin; treating or preventing photodamaged skin; reducing the signs of skin aging; reducing the appearance of hyperpigmentation; treating or preventing hyperpigmentation; treating or preventing pigment deposition in the skin (e.g., that caused by UV exposure); reducing the appearance of skin discolorations; and whitening, lightening, and/or bleaching the skin.

In certain embodiments, the compositions may be used for care, treatment, cleansing, and/or protective products for facial or body skin; anti-wrinkle or anti-aging compositions; skin firming compositions; skin lightening compositions; compositions for irritated skin; sunscreen compositions, artificial tanning (self-tanning) compositions or after-sun care compositions; scalp care compositions; shaving preparation compositions; depilatory compositions; or make-up products for the skin of the face or body.

In certain embodiments, the compositions comprising eriodictyol are administered for altering the aesthetic appearance of skin associated with or affected by, or treating or preventing, a skin condition/disorder (e.g., a skin condition/disorder accompanied with a loss of skin elasticity).

In certain embodiments, the compositions comprising eriodictyol are administered for improving the barrier function and viability of the skin.

In certain embodiments, the compositions comprising eriodictyol are administered for altering the aesthetic appearance of skin associated with or affected by wrinkling, sagging, and/or a loss of skin elasticity.

In certain embodiments, the compositions comprising eriodictyol are administered for altering the aesthetic appearance of skin associated with or affected by deteriorations in skin viscoelasticity.

In certain embodiments, the compositions comprising eriodictyol are administered for altering the aesthetic appearance of skin associated with or affected by one or more of wrinkles and/or fine lines, wizened skin, a lack of elasticity and/or of tonus of the skin, thinning of the dermis, degradation of collagen fibers, flaccid skin, thinned skin, and the internal degradation of the skin following exposure to ultraviolet radiation.

In certain embodiments, the compositions comprising eriodictyol are administered for decreasing the appearance of fine lines and/or wrinkles in the skin.

In certain embodiments, the compositions comprising eriodictyol are administered for decreasing the appearance of bags and/or rings around the eyes.

In certain embodiments, the compositions comprising eriodictyol are administered for reducing the appearance of hyperpigmentation.

In certain embodiments, the compositions comprising eriodictyol are administered for improving or increasing one or more of the thickness, elasticity, flexibility, radiance, glow, and plumpness of the skin.

In certain embodiments, the compositions comprising eriodictyol are administered for improving the fineness of skin texture.

In certain embodiments, the compositions comprising eriodictyol are administered for improving the appearance of wrinkled, lined, dry, flaky, aged or photodamaged skin.

In certain embodiments, the compositions comprising eriodictyol are administered for altering the aesthetic appearance of skin associated with or affected by skin discolorations.

### Methods of Administration:

In one aspect, the invention provides methods of administration of the compositions comprising eriodictyol. In certain embodiments, the invention the compositions of the invention are designed for topical administration.

In certain embodiments, the compositions of the invention may be administered as a topical composition on the skin of the subject. The compositions of the invention may be included in any formulation suitable for topical administration. In certain embodiments, the composition of the invention is a fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, and/or hair treatment.

In certain other embodiments, the compositions comprising eriodictyol may be prepared and used in the form of an aerosol spray, cream, emulsion, solid, liquid, dispersion, foam, oil, gel, lotion, mousse, ointment, powder, patch, pomade, solution, pump spray, stick, towelette, soap, or other forms commonly employed in the art of topical administration and/or cosmetic and skin care formulation. The compositions can be in an emulsion form. In addition, compounds provided herein used in the compositions provided herein can be used in color cosmetic compositions such as foundation makeups, blushes, eyeshadows, mascaras, concealers, eyeliners, lip colors, nail colors, and so on. Other cosmetic compositions can include perfumes, lipsticks, fingernail and toenail polish, eye and facial makeup, towelettes, deodorants, hand sanitizer, baby products, bath oils, bubble baths, and butters.

In certain embodiments, the compositions comprising eriodictyol are administered on the skin at a concentration of about 100 μg/cm² to about 5 mg/cm². In certain embodiments, the compositions comprising eriodictyol are administered on the skin at a concentration of about 200 μg/cm² to about 4 mg/cm². In certain embodiments, the compositions comprising eriodictyol are administered on the skin at a concentration of about 400 μg/cm² to about 3 mg/cm². In certain embodiments, the compositions comprising eriodictyol are administered on the skin at a concentration of about 1 mg μg/cm² to about 2 mg/cm². In certain embodiments, the compositions comprising eriodictyol are administered on the skin at a concentration of about 2 mg μg/cm².

In certain embodiments, the composition comprising eriodictyol is administered on the affected area of the skin. The affected area of skin is the area of skin to be improved or otherwise suffering from a topical condition. In certain embodiments, the compositions comprising eriodictyol are administered over an area of about 0.5 cm² to about 100 cm². In certain embodiments, the compositions comprising eriodictyol are administered over an area of about 1 cm² to about 75 cm². In certain embodiments, the compositions comprising eriodictyol are administered over an area of about 10 cm² to about 75 cm². In certain embodiments, the compositions comprising eriodictyol are administered over an area of about 50 cm².

In certain embodiments, the compositions comprising eriodictyol are administered for a duration till the desired impact in amelioration or prevention of a topical condition is realized. In certain embodiments, the composition comprising eriodictyol comprises an effective amount of eriodictyol The effective amount of eriodictyol is an amount of eriodictyol that results in the desired result in the condition of the skin. In certain embodiments, the composition comprising eriodictyol is administered once a day. In certain embodiments, the composition comprising eriodictyol is administered twice a day. In certain embodiments, the composition comprising eriodictyol is administered thrice a day. In certain embodiments, the composition comprising eriodictyol is administered once every two days. In certain embodiments, the composition comprising eriodictyol is administered once every three days.

### EXAMPLES

### Example 1: Exemplary Composition Comprising Eriodictyol

Table 4 provides an exemplary composition comprising eriodictyol.

TABLE 4
Exemplary composition comprising Eriodictyol

| | Concentration |
|---|---|
| Ingredient | % w/w |
| Water | 76.300 |
| Glycerin | 5.000 |
| Propanediol | 2.000 |
| Hydrogenated Ethylhexyl Olivate | 0.500 |
| Hydrogenated Olive Oil Unsaponifiables | 0.500 |
| Polyglyceryl-6 Distearate | 1.419 |
| Jojoba Esters | 0.407 |
| Polyglyceryl-3 Beeswax | 0.187 |
| Cetyl Alcohol | 0.187 |
| Cetearyl Olivate | 1.000 |
| Sorbitan Olivate | 1.000 |
| Hydroxyethyl Acrylate/Sodium Acryloyldimethyl Taurate | 1.500 |
| Copolymer | |
| Helianthus Annuus (Sunflower) Seed Oil | 3.000 |
| Caprylic/Capric Triglyceride | 3.000 |
| Phenoxyethanol | 0.700 |
| Decylene Glycol | 0.225 |
| 1,2-Hexanediol | 0.075 |
| Eriodictyol | 0.500 |
| Dimethyl Isosorbide | 2.500 |

### Example 2: ABTS Antioxidant Assay

The 2,2′-Azinobis [3-ethylbenzothiazoline-6-sulfonic acid]-diammonium salt (ABTS) antioxidant assay is designed to evaluate the antioxidant activity of compositions comprising eriodictyol in an aqueous medium.

ABTS+ solution is generated as a free radical and used to measure relative ability of a compound to scavenge free radicals. Reduction of the absorbance at 734 nm was standardized to solvent control as an indication of ABTS+ scavenging.

Table 5, provided below, demonstrates the results from these experiments:

TABLE 5
ABTS (aqueous antioxidant assay) radical inhibition

| Eriodictyol | % Radical |
|---|---|
| concentration (% w/w) | Inhibition |
| 0.004 | 92.97 |
| 0.0008 | 58.72 |
| 0.00016 | 11.00 |

As evident from the results provided in Table 5 above, compositions comprising eriodictyol demonstrate antioxidant activity at concentrations at low as 0.00016% w/w in aqueous systems. The calculated IC₅₀ value is: 0.00062% w/w.

### Example 3: DPPH Antioxidant Activity Assay

The 2,2-diphenyl-1-picrylhydrazyl (DPPH) antioxidant assay is designed to evaluate the antioxidant activity of compositions comprising eriodictyol in an organic medium.

2,2-diphenyl-1-picrylhydrazyl (DPPH) is a stable free radical used to evaluate free radical scavenging capacity of a material. In the presence of an antioxidant DPPH is reduced, resulting in the formation of a colorless solution. Reduction of absorbance at 517 nm is standardized to solvent control as an indication of DPPH scavenging.

Table 6, provided below, provides the antioxidant activity data.

TABLE 6
DPPH Radical Inhibition

| Eriodictyol | % Radical |
|---|---|
| concentration (% w/w) | Inhibition |
| 0.00278 | 81.95 |
| 0.00046 | 81.45 |
| 0.00007 | 21.22 |

As evident from the results provided in Table 6 above, compositions comprising eriodictyol demonstrate antioxidant activity at concentrations at low as 0.00007% w/w in organic solvent systems. The calculated IC₅₀ value is: 0.0002% w/w.

### Example 4: Elastase Enzyme Inhibition

The assay was conducted to evaluate the elastase enzyme inhibition activity of compositions comprising eriodictyol.

Neutrophil infiltration and neutrophil elastase are both increased in the skin in response to UV stress. Neutrophil elastase has been shown to activate MMP-1 and MMP-2 collagenase in response to low dose UV exposure, harming the extracellular matrix and contributing to photoaging and wrinkle formation. The activity of elastase can be further upregulated in the presence of reactive oxygen species. Therefore, inhibition of this enzyme, particularly in combination with antioxidant activity, is effective in delaying wrinkle development and aiding in maintaining skin integrity. This assay is measures release of 4-nitroaniline from an Me-Suc-Ala-Ala-Pro-Val-pNA by human neutrophil elastase and can be quantified at 405 nm by comparing to solvent control.

Table 7 provides the elastase inhibition activity of compositions comprising eriodictyol:

TABLE 7
Elastase enzyme inhibition

| Eriodictyol | % Enzyme |
|---|---|
| concentration (% w/w) | Inhibition |
| 0.2 | 58.08 |
| 0.04 | 23.12 |
| 0.008 | 17.56 |

As evident from the data provide in Table 7, compositions comprising eriodictyol inhibits elastase enzyme activity as low at 0.008% w/w. The calculated IC₅₀ value is: 0.107% w/w.

### Example 5: Glycation Inhibition Assay

The assay was conducted to evaluate the glycation inhibition activity of compositions comprising eriodictyol.

Nonenzymatic glycation can occur when glucose reacts with proteins, forming a Schiff base intermediate that subsequently undergoes Amadori rearrangement to form glycosylated proteins, when they react with skin proteins to form Advanced Glycation End products (AGE). AGE formation can result in loss of skin elasticity and impaired cellular function. The glycation inhibition ability of a compound is measured by change in fluorescence at excitation/emission of 360 nm/420 nm relative to solvent control.

Table 8 provides the data for glycation inhibition.

TABLE 8
Glycation Inhibition

| Eriodictyol | % Enzyme |
|---|---|
| concentration (% w/w) | Inhibition |
| 0.022 | 90.26 |
| 0.007 | 86.18 |
| 0.002 | 38.43 |

As evident from the data provided in Table 7, compositions comprising eriodictyol inhibits glycation as low as 0.002% w/w. The IC₅₀ value calculated for glycation inhibition is 0.0088% w/w.

### Example 6: Gene Modulation

Table 9, provided below, incorporates the gene modulation data for formulations comprising eriodictyol.

| Gene | | | Fold | | |
|---|---|---|---|---|---|
| Name | Treatment | Control | Change | P value | Gene Description |
| MMP10 | 106.9 | 2653.7 | −24.3 | 3.7e−21 | matrix metallopeptidase |
| | | | | | 10 |
| PPFIA4 | 628.6 | 22.1 | 27.9 | 8.6e−20 | PTPRF interacting protein |
| | | | | | alpha 4 |
| THBS1 | 344.3 | 5670.9 | −16.0 | 6.8e−18 | thrombospondin 1 |
| TOP2A | 37.6 | 697.9 | −18.4 | 1.3e−16 | DNA topoisomerase II |
| | | | | | alpha |
| TFPI2 | 34.1 | 595.8 | −17.1 | 6.5e−16 | tissue factor pathway |
| | | | | | inhibitor 2 |
| MMP1 | 972.9 | 11100.1 | −11.3 | 9.8e−15 | matrix metallopeptidase 1 |
| FN1 | 146.9 | 1783.9 | −12.1 | 1.3e−14 | fibronectin 1 |
| KCTD4 | 36.4 | 542.1 | −14.9 | 1.7e−14 | potassium channel |
| | | | | | tetramerization domain |
| | | | | | containing 4 |
| ATP12A | 76.4 | 879.2 | −11.3 | 1.8e−13 | ATPase H+/K+ |
| | | | | | transporting non-gastric |
| | | | | | alpha2 subunit |
| ALDH1A3 | 168.0 | 1707.3 | −9.8 | 4.0e−13 | aldehyde dehydrogenase |
| | | | | | 1 family member A3 |
| MKI67 | 78.7 | 828.1 | −10.6 | 9.3e−13 | marker of proliferation |
| | | | | | Ki-67 |
| FST | 96.4 | 980.5 | −9.8 | 1.2e−12 | follistatin |
| SLC13A5 | 5.9 | 85.9 | −13.9 | 1.7e−08 | Solute carrier family 13 |
| | | | | | member 5 |
| IL6 | 1.2 | 25.5 | −19.7 | 1.2e−04 | Interleukin 6 |
| IL23A | 5.9 | 35.7 | −6.1 | 3.9e−04 | Interleukin 23 |
| TNF | 0.0 | 31.5 | −256.0 | 2.4e−07 | Tumor Necrosis Factor |
| IL24 | 1.2 | 95.3 | −73.5 | 2.2e−12 | Interleukin 24 |
| MMP3 | 8.2 | 399.2 | −48.5 | 3.5e−20 | Matrix Metallopeptidase |
| | | | | | 3 |
| MMP9 | 527.6 | 4547.5 | −8.6 | 4.3e−12 | Matrix Metallopeptidase |
| | | | | | 9 |

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A composition for topical administration comprising an effective amount of eriodictyol.

2. The composition of claim 1, wherein the composition comprises an emulsion.

3. The composition of claim 2, wherein the emulsion is an oil/water emulsion.

4. The composition of claim 1, wherein the composition further comprises a solvent.

5. The composition of claim 4, wherein the solvent is a cosmetic solvent.

6. The composition of claim 5, wherein the solvent is selected from a group consisting of: dimethyl isosorbide, ethoxydiglycol, isopropyl lauroyl sarcosinate, and any combinations thereof.

7. The composition of claim 6, wherein the solvent is dimethyl isosorbide.

8. The composition of claim 6, wherein the solvent is ethoxydiglycol.

9. The composition of claim 6, wherein the solvent is isopropyl lauroyl sarcosinate.

10. The composition of claim 1, which is stable under storage for at least two (2) years under ambient conditions.

11. The composition of claim 1, which is stable under storage for at least one (1) year under ambient conditions.

12. The composition of claim 1, which is stable under storage for at least six (6) months under ambient conditions.

13. The composition of claim 1, which is stable under storage for at least three (3) months under ambient conditions.

14. The composition of claim 1, wherein the composition demonstrates less than about 5% degradation when stored at 50° C. for one (1) month.

15. The composition of claim 1, wherein the composition demonstrates less than about 10% degradation when stored at 50° C. for one (1) month.

16. The composition of claim 1, wherein the composition demonstrates less than about 10% degradation when stored at 40° C. for one (1) month.

17. The composition of claim 1, wherein the composition demonstrates less than about 1% degradation when stored at 25° C. and/or 4° C. for one (1) month.

18. The composition of claim 1, wherein the composition demonstrates less than about 10% degradation when stored at 40° C. for three (3) months.

19. The composition of claim 1, wherein the composition demonstrates less than about 10% degradation when stored at 25° C. for three (3) months.

20. The composition of claim 1, wherein the composition demonstrates less than about 10% degradation when stored at 25° C. for six (6) months.

21. The composition of claim 1, wherein the composition demonstrates less than about 5% degradation when stored at 4° C. for three (3) months.

22. The composition of claim 1, wherein the pH of the composition is from about 4 to about 8.

23. The composition of claim 1, wherein the pH of the composition is from about 4 to about 7.

24. The composition of claim 1, wherein the pH of the composition is from about 4 to about 6.

25. The composition of claim 1, wherein the pH of the composition is from about 4 to about 5.

26. The composition of claim 1, wherein the composition of pH is about 4.

27. The composition of claim 26, wherein the composition demonstrates less than about 10% degradation when stored at 50° C. for one (1) month.

28. The composition of claim 1, wherein the composition of pH is about 5.

29. The composition of claim 28, wherein the composition demonstrates less than about 10% degradation when stored at 50° C. for one (1) month.

30. The composition of claim 1, wherein the composition of pH is about 6.

31. The composition of claim 30, wherein the composition demonstrates less than about 10% degradation when stored at 50° C. for one (1) month.

32. The composition of claim 1, wherein the composition of pH is about 7.

33. The composition of claim 32, wherein the composition demonstrates less than about 10% degradation when stored at 50° C. for one (1) month.

34. The composition of claim 1, wherein eriodictyol is present in a concentration of about 0.0001% to about 5.0% w/w.

35. The composition of claim 1, wherein eriodictyol is present in a concentration of about 0.0001% to about 1.0% w/w.

36. The composition of claim 1, wherein eriodictyol is present in a concentration of about 0.001% to about 0.5% w/w.

37. The composition of claim 1, wherein eriodictyol is present in a concentration of about 0.01% to about 0.5% w/w.

38. The composition of claim 1, wherein eriodictyol is present in a concentration of about 0.1% to about 0.5% w/w.

39. The composition of claim 1, wherein eriodictyol is present in a concentration of about 0.5% w/w.

40. The composition of claim 1, wherein the composition is selected from a group consisting of: fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, hair treatment, and any combination thereof.

41. The composition of claim 1, wherein the composition further comprises one or more excipients selected from a group consisting of: solvent, emulsifier, preservative, antioxidant, emollient, thickening agent, penetration enhancer, surfactant, diluent, filler, carrier, and/or pH control agent.

42. The composition of claim 41, wherein the preservative is an antimicrobial preservative or chelating agent.

43. The composition of claim 1, wherein eriodictyol is encapsulated.

44. The composition of claim 1, wherein the composition further comprises one or more excipients selected from a group consisting of: dimethyl isosorbide, ethoxydiglycol, water, glycerin, propanediol, hydrogenated ethylhexyl olivate, hydrogenated olive oil unsaponifiables, polyglycerol-6-distearate, jojoba esters, polyglyceryl-3-beeswax, cetyl alcohol, cetearyl olivate, sorbitan olivate, hydroxyethyl acrylate/sodium acryloyldimethyl taurate copolymer, Helianthus annuus (sunflower) seed oil, caprylic/capric triglyceride, phenoxyethanol, decylene glycol, and 1,2-hexanediol.

45. The composition of claim 1, wherein the composition is:

| | Concentration |
|---|---|
| Ingredient | % w/w |
| Water | 30-90 |
| Glycerin | 0-20 |
| Propanediol | 0-10 |
| Hydrogenated Ethylhexyl Olivate | 0-2 |
| Hydrogenated Olive Oil Unsaponifiables | 0-2 |
| Polyglyceryl-6 Distearate | 0-5 |
| Jojoba Esters | 0-2.5 |
| Polyglyceryl-3 Beeswax | 0-2 |
| Cetyl Alcohol | 0-2 |
| Cetearyl Olivate | 0-5 |
| Sorbitan Olivate | 0-5 |
| Hydroxyethyl Acrylate/Sodium Acryloyldimethyl Taurate | 0-5 |
| Copolymer | |
| Helianthus Annuus (Sunflower) Seed Oil | 0-10 |
| Caprylic/Capric Triglyceride | 0-10 |
| Phenoxyethanol | 0-2 |
| Decylene Glycol | 0-2 |
| 1,2-Hexanediol | 0-2 |
| Eriodictyol | 0.0001-5 |
| Dimethyl Isosorbide | 0-10 |

46. The composition of claim 1, wherein the composition is:

| | Concentration |
|---|---|
| Ingredient | % w/w |
| Water | 76.300 |
| Glycerin | 5.000 |
| Propanediol | 2.000 |
| Hydrogenated Ethylhexyl Olivate | 0.500 |
| Hydrogenated Olive Oil Unsaponifiables | 0.500 |
| Polyglyceryl-6 Distearate | 1.419 |
| Jojoba Esters | 0.407 |
| Polyglyceryl-3 Beeswax | 0.187 |
| Cetyl Alcohol | 0.187 |
| Cetearyl Olivate | 1.000 |
| Sorbitan Olivate | 1.000 |
| Hydroxyethyl Acrylate/Sodium Acryloyldimethyl Taurate | 1.500 |
| Copolymer | |
| Helianthus Annuus (Sunflower) Seed Oil | 3.000 |
| Caprylic/Capric Triglyceride | 3.000 |
| Phenoxyethanol | 0.700 |
| Decylene Glycol | 0.225 |
| 1,2-Hexanediol | 0.075 |
| Eriodictyol | 0.500 |
| Dimethyl Isosorbide | 2.500 |

47. A method of treating or preventing a topical condition in a subject by administration of a composition comprising an effective amount of eriodictyol.

48. The method of claim 47, wherein the administration of the composition prevents fine lines or wrinkles on skin of the subject.

49. The method of claim 47, wherein the administration of the composition leads to supporting firmness and elasticity of skin of the subject.

50. The method of claim 47, wherein the administration of the composition leads to the prevention of skin and hair damaged by reactive oxygen species.

51. The method of claim 47, wherein the administration of the composition leads to protection from environment aggressors on skin.

52. The method of claim 47, wherein the administration of the composition leads to supporting skin elasticity.

53. The method of claim 47, wherein the topical condition is glycation.

54. The method of claim 47, wherein the administration of the composition leads to increase in skin and/or hair resiliency.

55. The method of claim 47, wherein the administration of the composition leads to increase in elasticity of skin.

56. The method of claim 47, wherein the administration of the composition leads to prevention of skin damage.

57. The method of claim 47, wherein the composition is administered topically.

58. The method of claim 47, wherein the composition comprises an emulsion.

59. The method of claim 58, wherein the emulsion is an oil/water emulsion.

60. The method of claim 47, wherein the composition further comprises a solvent.

61. The method of claim 60, wherein the solvent is selected from a group consisting of: dimethyl isosorbide, ethoxydiglycol, isopropyl lauroyl sarcosinate, and any combinations thereof.

62. The method of claim 58, wherein the solvent is dimethyl isosorbide.

63. The method of claim 58, wherein the solvent is ethoxydiglycol.

64. The method of claim 58, wherein the solvent is isopropyl lauroyl sarcosinate.

65. The method of claim 47, wherein the composition is stable under storage for at least two (2) years under ambient conditions.

66. The method of claim 47, wherein the composition is stable under storage for at least one (1) year under ambient conditions.

67. The method of claim 47, wherein the composition is stable under storage for at least six (6) months under ambient conditions.

68. The method of claim 47, wherein the composition is stable under storage for at least three (3) months under ambient conditions.

69. The method of claim 47, wherein the composition demonstrates less than about 5% degradation when stored at 50° C. for one (1) month.

70. The method of claim 47, wherein the composition demonstrates less than about 10% degradation when stored at 50° C. for one (1) month.

71. The method of claim 47, wherein the composition demonstrates less than about 10% degradation when stored at 40° C. for one (1) month.

72. The method of claim 47, wherein the composition demonstrates less than about 1% degradation when stored at 25° C. and/or 4° C. for one (1) month.

73. The method of claim 47, wherein the composition demonstrates less than about 10% degradation when stored at 40° C. for three (3) months.

74. The method of claim 47, wherein the composition demonstrates less than about 10% degradation when stored at 25° C. for three (3) months.

75. The method of claim 47, wherein the composition demonstrates less than about 10% degradation when stored at 25° C. for six (6) months.

76. The method of claim 47, wherein the composition demonstrates less than about 5% degradation when stored at 4° C. for three (3) months.

77. The method of claim 47, wherein the pH of the composition is from about 4 to about 8.

78. The method of claim 47, wherein the pH of the composition is from about 4 to about 7.

79. The method of claim 47, wherein the pH of the composition is from about 4 to about 6.

80. The method of claim 47, wherein the pH of the composition is from about 4 to about 5.

81. The method of claim 47, wherein the composition of pH is about 4.

82. The method of claim 81, wherein the composition demonstrates less than about 10% degradation when stored at 50° C. for one (1) month.

83. The method of claim 47, wherein the composition of pH is about 5.

84. The method of claim 83, wherein the composition demonstrates less than about 10% degradation when stored at 50° C. for one (1) month.

85. The method of claim 47, wherein the composition of pH is about 6.

86. The method of claim 85, wherein the composition demonstrates less than about 10% degradation when stored at 50° C. for one (1) month.

87. The method of claim 47, wherein the composition of pH is about 7.

88. The method of claim 87, wherein the composition demonstrates less than about 10% degradation when stored at 50° C. for one (1) month.

89. The method of claim 47, wherein eriodictyol is present in a concentration of about 0.0001% to about 5.0% w/w.

90. The method of claim 47, wherein eriodictyol is present in a concentration of about 0.0001% to about 1.0% w/w.

91. The method of claim 47, wherein eriodictyol is present in a concentration of about 0.001% to about 0.5% w/w.

92. The method of claim 47, wherein eriodictyol is present in a concentration of about 0.01% to about 0.5% w/w.

93. The method of claim 47, wherein eriodictyol is present in a concentration of about 0.1% to about 0.5% w/w.

94. The method of claim 47, wherein eriodictyol is present in a concentration of about 0.5% w/w.

95. The method of claim 47, wherein the composition is selected from a group consisting of: fluid, emulsion, encapsulation, suspension, solid, semi-solid, jelly, paste, gel, hydrogel, ointment, lotion, emulsion, cream, foam, mousse, liquid, spray, suspension, dispersion, powder, aerosol, color cosmetic, hair treatment, and any combination thereof.

96. The method of claim 47, wherein the composition further comprises one or more excipients selected from a group consisting of: solvent, emulsifier, preservative, antioxidant, emollient, thickening agent, penetration enhancer, surfactant, diluent, filler, carrier, and/or pH control agent.

97. The method of claim 96, wherein the preservative is an antimicrobial preservative or chelating agent.

98. The method of claim 47, wherein the eriodictyol is encapsulated in the composition.

99. The method of claim 47, wherein the composition further comprises one or more excipients selected from a group consisting of: dimethyl isosorbide, ethoxydiglycol, water, glycerin, propanediol, hydrogenated ethylhexyl olivate, hydrogenated olive oil unsaponifiables, polyglycerol-6-distearate, jojoba esters, polyglyceryl-3-beeswax, cetyl alcohol, cetearyl olivate, sorbitan olivate, hydroxyethyl acrylate/sodium acryloyldimethyl taurate copolymer, Helianthus annuus (sunflower) seed oil, caprylic/capric triglyceride, phenoxyethanol, decylene glycol, and 1,2-hexanediol.

100. The method of claim 47, wherein the composition is:

| | Concentration |
|---|---|
| Ingredient | % w/w |
| Water | 30-90 |
| Glycerin | 0-20 |
| Propanediol | 0-10 |
| Hydrogenated Ethylhexyl Olivate | 0-2 |
| Hydrogenated Olive Oil Unsaponifiables | 0-2 |
| Polyglyceryl-6 Distearate | 0-5 |
| Jojoba Esters | 0-2.5 |
| Polyglyceryl-3 Beeswax | 0-2 |
| Cetyl Alcohol | 0-2 |
| Cetearyl Olivate | 0-5 |
| Sorbitan Olivate | 0-5 |
| Hydroxyethyl Acrylate/Sodium Acryloyldimethyl Taurate | 0-5 |
| Copolymer | |
| Helianthus Annuus (Sunflower) Seed Oil | 0-10 |
| Caprylic/Capric Triglyceride | 0-10 |
| Phenoxyethanol | 0-2 |
| Decylene Glycol | 0-2 |
| 1,2-Hexanediol | 0-2 |
| Eriodictyol | 0.0001-5 |
| Dimethyl Isosorbide | 0-10 |

101. The method of claim 47, wherein the composition is:

| | Concentration |
|---|---|
| Ingredient | % w/w |
| Water | 76.300 |
| Glycerin | 5.000 |
| Propanediol | 2.000 |
| Hydrogenated Ethylhexyl Olivate | 0.500 |
| Hydrogenated Olive Oil Unsaponifiables | 0.500 |
| Polyglyceryl-6 Distearate | 1.419 |
| Jojoba Esters | 0.407 |
| Polyglyceryl-3 Beeswax | 0.187 |
| Cetyl Alcohol | 0.187 |
| Cetearyl Olivate | 1.000 |
| Sorbitan Olivate | 1.000 |
| Hydroxyethyl Acrylate/Sodium Acryloyldimethyl Taurate | 1.500 |
| Copolymer | |
| Helianthus Annuus (Sunflower) Seed Oil | 3.000 |
| Caprylic/Capric Triglyceride | 3.000 |
| Phenoxyethanol | 0.700 |
| Decylene Glycol | 0.225 |
| 1,2-Hexanediol | 0.075 |
| Eriodictyol | 0.500 |
| Dimethyl Isosorbide | 2.500 |

102. A method of modulating expression of one or more genes by administration of a composition comprising eriodictyol, wherein at least one of the genes is related to a topical condition.

103. The method of claim 102, wherein the one more genes are selected from a group consisting of: MMP10, THBS1, TOP2A, TFPI2, MMP1, FN1, KCTD4, ATP12A, ALDH1A3, MKI67, FST, SLC13A5, IL6, IL23A, TNF, IL24, MMP3, MMP9, and/or PPFIA4.

104. The method of claim 102, wherein the administration of the composition leads to down-regulation of expression and/or activity of MMP10, THBS1, TOP2A, TFPI2, MMP1, FN1, KCTD4, ATP12A, ALDH1A3, MKI67, FST, SLC13A5, IL6, IL23A, TNF, IL24, MMP3, and/or MMP9.

105. The method of claim 102, wherein the administration of the composition leads to up-regulation of expression and/or activity of PPFIA4.

106. The method of claim 102, wherein the administration of the composition enhances anti-inflammatory activity.

107. The method of claim 106, wherein the administration of the composition results in downregulation of expression and/or activity of IL6, IL23a, TNF, and/or IL24.

108. The method of claim 102, wherein the administration of the composition leads to anti-aging activity.

109. The method of claim 108, wherein the administration of the composition results in downregulation of expression and/or activity of MMP10, MMP9, MMP3, and MMP1.

110. The method of claim 102, wherein the administration of the composition enhances maintenance of skin barrier lipids.

111. The method of claim 110, wherein the administration of the composition results in upregulation of expression and/or activity of PPFIA4.

112. The method of claim 102, wherein the administration of the composition results in prevention of formation of scar tissue after healing of wounds.

113. The method of claim 112, wherein the administration of the composition leads to downregulation of expression and/or activity of THBS1.

114. The method of claim 102, wherein the composition is administered topically.

115. The method of claim 114, wherein the topical administration is undertaken on the skin of the patient.

116. The method of claim 102, wherein the administration of the composition leads to at least about ten (10) fold downregulation of the activity and/or expression of matrix metallopeptidase 10 (MMP10).

117. The method of claim 102, wherein the administration of the composition leads to about twenty-five (25) fold downregulation of the activity and/or expression of matrix metallopeptidase 10 (MMP10).

118. The method of claim 102, wherein the administration of the composition leads to at least about ten (10) fold downregulation of the activity and/or expression of thrombospondin 1 (THBS1).

119. The method of claim 102, wherein the administration of the composition leads to about fifteen (15) fold downregulation of the activity and/or expression of thrombospondin 1 (THBS1).

120. The method of claim 102, wherein the administration of the composition leads to at least about ten (10) fold downregulation of the activity and/or expression of DNA topoisomerase II-α (TOP2A).

121. The method of claim 102, wherein the administration of the composition leads to about fifteen (15) fold downregulation of the activity and/or expression of DNA topoisomerase II-α (TOP2A).

122. The method of claim 102, wherein the administration of the composition leads to at least about ten (10) fold downregulation of the activity and/or expression of tissue factor pathway inhibitor 2 (TFPI2).

123. The method of claim 102, wherein the administration of the composition leads to about fifteen (15) fold downregulation of the activity and/or expression of tissue factor pathway inhibitor 2 (TFPI2).

124. The method of claim 102, wherein the administration of the composition leads to at least about two (2) fold downregulation of the activity and/or expression of matrix metallopeptidase 1 (MMP1).

125. The method of claim 102, wherein the administration of the composition leads to about ten (10) fold downregulation of the activity and/or expression of matrix metallopeptidase 1 (MMP1).

126. The method of claim 102, wherein the administration of the composition leads to at least about two (2) fold downregulation of the activity and/or expression of fibronectin 1 (FN1).

127. The method of claim 102, wherein the administration of the composition leads to about ten (10) fold downregulation of the activity and/or expression of fibronectin 1 (FN1).

128. The method of claim 102, wherein the administration of the composition leads to at least about five (5) fold downregulation of the activity and/or expression of potassium channel tetramerization domain containing 4 (KCTD4).

129. The method of claim 102, wherein the administration of the composition leads to about fifteen (15) fold downregulation of the activity and/or expression of potassium channel tetramerization domain containing 4 (KCTD4).

130. The method of claim 102, wherein the administration of the composition leads to at least about two (2) fold downregulation of the activity and/or expression of ATPase H+/K+ transporting non-gastric α2 subunit (ATP12A).

131. The method of claim 102, wherein the administration of the composition leads to about ten (10) fold downregulation of the activity and/or expression of ATPase H+/K+ transporting non-gastric α2 subunit (ATP12A).

132. The method of claim 102, wherein the administration of the composition leads to at least about two (2) fold downregulation of the activity and/or expression of aldehyde dehydrogenase 1 family member A3 (ALDH1A3).

133. The method of claim 102, wherein the administration of the composition leads to about ten (10) fold downregulation of the activity and/or expression of aldehyde dehydrogenase 1 family member A3 (ALDH1A3).

134. The method of claim 102, wherein the administration of the composition leads to at least about two (2) fold downregulation of the activity and/or expression of marker of proliferation Ki-67 (MKI67).

135. The method of claim 102, wherein the administration of the composition leads to about ten (10) fold downregulation of the activity and/or expression of marker of proliferation Ki-67 (MKI67).

136. The method of claim 102, wherein the administration of the composition leads to at least about two (2) fold downregulation of the activity and/or expression of follistatin (FST).

137. The method of claim 102, wherein the administration of the composition leads to about ten (10) fold downregulation of the activity and/or expression of follistatin (FST).

138. The method of claim 102, wherein the administration of the composition leads to at least about five (5) fold downregulation of the activity and/or expression of solute carrier family 13 member 5 (SLC13A5).

139. The method of claim 102, wherein the administration of the composition leads to about fifteen (15) fold downregulation of the activity and/or expression of solute carrier family 13 member 5 (SLC13A5).

140. The method of claim 102, wherein the administration of the composition leads to at least about five (5) fold downregulation of the activity and/or expression of interleukin 6 (IL6).

141. The method of claim 102, wherein the administration of the composition leads to about twenty (20) fold downregulation of the activity and/or expression of interleukin 6 (IL6).

142. The method of claim 102, wherein the administration of the composition leads to at least about two (2) fold downregulation of the activity and/or expression of interleukin 23 (IL23).

143. The method of claim 102, wherein the administration of the composition leads to about five (5) fold downregulation of the activity and/or expression of interleukin 23 (IL23).

144. The method of claim 102, wherein the administration of the composition leads to at least about ten (10) fold downregulation of the activity and/or expression of tumor necrosis factor (TNF).

145. The method of claim 102, wherein the administration of the composition leads to about two hundred fifty (250) fold downregulation of the activity and/or expression of tumor necrosis factor (TNF).

146. The method of claim 102, wherein the administration of the composition leads to at least about ten (10) fold downregulation of the activity and/or expression of interleukin 24 (IL24).

147. The method of claim 102, wherein the administration of the composition leads to about seventy (70) fold downregulation of the activity and/or expression of interleukin 24 (IL24).

148. The method of claim 102, wherein the administration of the composition leads to at least about ten (10) fold downregulation of the activity and/or expression of matrix metallopeptidase 3 (MMP3).

149. The method of claim 102, wherein the administration of the composition leads to about fifty (50) fold downregulation of the activity and/or expression of matrix metallopeptidase 3 (MMP3).

150. The method of claim 102, wherein the administration of the composition leads to at least about two (2) fold downregulation of the activity and/or expression of matrix metallopeptidase 9 (MMP9).

151. The method of claim 102, wherein the administration of the composition leads to about eight (8) fold downregulation of the activity and/or expression of matrix metallopeptidase 9 (MMP9).

152. The method of claim 102, wherein the administration of the composition leads to about five (5) fold upregulation of the activity and/or expression of stimulated by PTPRF interacting protein α4 (PPFIA4).

153. The method of claim 102, wherein the administration of the composition leads to about thirty (30) fold upregulation of the activity and/or expression of PTPRF interacting protein α4 (PPFIA4).

154. The method of claim 102, wherein the administration of the composition to a subject leads to the treatment of prevention of a topical condition of the subject.

155. The method of claim 102, wherein the administration of composition alters one or more of a function related with the modulated genes, wherein the one or more function is selected from the group consisting of: (i) maintenance of skin barrier, (ii) anti-aging, (iii) inflammation, (iv) prevention of scar formation upon wound healing, and (v) maintenance of skin barrier lipids.
